(19) United States
(12) Patent Application Publication
(10) Pub. No.: US 2021/0198187 A1
(43) Pub. Date: Jul. 1, 2021

(54) PROCESS FOR SEPARATING TRICYANOHEXANE

(71) Applicant: Ascend Performance Materials Operations LLC, Houston, TX (US)

(72) Inventors: Sanjay Dube, Madison, AL (US); Benjamin Haseltine, Houston, TX (US); Jefferson Thomas Ebert, League City, TX (US); Darrick Elmore, Pensacola, FL (US)

(73) Assignee: Ascend Performance Materials Operations LLC, Houston, TX (US)

(21) Appl. No.: 17/138,605

(22) Filed: Dec. 30, 2020

Related U.S. Application Data
(60) Provisional application No. 62/955,066, filed on Dec. 30, 2019.

(51) Int. Cl. C07C 253/34; C07C 255/05; B01D 15/20; B01D 15/24; B01D 15/18

(52) U.S. Cl. CPC C07C 253/34; C07C 255/05; B01D 15/1864; B01D 15/24; B01D 15/206

(57) ABSTRACT

A process for producing a TCH stream, the process comprising: separating, in a first column, an adiponitrile process stream comprising TCH and optionally adiponitrile, to form an adiponitrile stream comprising greater than 5 wt. % adiponitrile and a first TCH stream comprising TCH, and optionally a heavies stream comprising high-boiling components and solid impurities; and optionally purifying the first TCH stream, via one or more columns, to form a purified TCH stream comprising greater than 50 wt. % TCH; wherein the first column is operated at a pressure drop less than 25 mmHg.

### REFERENCE TO RELATED APPLICATION

This application claims priority to U.S. Provisional Application No. 62/955,066, filed on Dec. 30, 2019, which is incorporated herein by reference.

### FIELD

The present disclosure relates generally to production of tricyanohexane (TCH) via purification of process streams of industrial processes. More specifically, the present disclosure relates to processes for producing high purity TCH from adiponitrile production process streams.

### BACKGROUND

Cyanocarbons, e.g., organic compounds having cyano functional groups, are known and are widely used in various applications. Many of these compounds, including acrylonitrile and adiponitrile, are used as monomers to prepare various polymers, such as nylon, polyacrylonitrile, or acrylonitrile butadiene styrene. Adiponitrile, in particular, can be hydrogenated to 1,6-diaminohexane (hexamethylenediamine (HMD)) for the production of nylon-6,6. Several methods of producing cyanocarbons are known in the art. For example, a conventional method of producing adiponitrile is the electrohydrodimerization of acrylonitrile, as described in U.S. Pat. No. 3,844,911. This and other production methods often yield streams comprising small amounts of desirable co-products. For example, some of the conventional streams of adiponitrile production processes may contain small but not insignificant amounts of TCH. TCH has a number of uses, including as a precursor for a number of industrial products or as an additive in lithium ion battery applications. Typically, separation of these streams has been inefficient and has not been able to effectively capture these amounts of TCH. As a result, the streams are treated as waste streams, e.g., burned. Accordingly, valuable TCH goes uncaptured.

The usefulness of TCH is described in a variety of references. One example is U.S. Pat. No. 7,262,256, which discloses a polycarboxylic acid mixture comprising 80% by weight or more of 1,3,6-hexanetricarboxylic acid, wherein the polycarboxylic acid mixture has a psychometric lightness L-value of 98 or more, a psychometric chroma a-value of from −2.0 to 2.0 and a psychometric chroma b-value of from −2.0 to 3.0, and has a nitrogen content of 5,000 ppm by weight or less. In particular, the polycarboxylic acid mixture is obtained from a hydrolysis reaction mixture obtained by hydrolyzing a nitrile mixture comprised mainly of 1,3,6-tricyanohexane.

Another example is US Publication No. 2013/0157119, which discloses a secondary battery in which decomposition of an electrolyte liquid is suppressed and generation of a gas is reduced, even in the case of using a laminate film as a package. The secondary batteries disclosed therein are of the stacked laminate type and comprise an electrode assembly in which a positive electrode and a negative electrode are arranged to face each other, an electrolyte liquid and a package accommodating the electrode assembly and said electrolyte liquid, wherein the negative electrode is formed by binding a negative electrode active substance comprising a metal (a) capable of being alloyed with lithium, a metal oxide (b) capable of occluding and releasing lithium ions and a carbon material (c) capable of occluding and releasing lithium ions, to a negative electrode current collector, with at least one selected from polyimides and polyamideimides, and the electrolyte liquid comprises a predetermined nitrile compound. In particular, electrolyte liquids containing 1,3,6-hexanetricarbonitrile are disclosed.

In view of these and other conventional uses for TCH, the need exists for cost-effective processes for recovering TCH. In particular, the need exists for processes for effectively recovering high purity TCH from adiponitrile production process streams comprising lower amounts thereof, thus capturing the TCH that is conventionally wasted.

### SUMMARY

In some embodiments, the present disclosure relates to a process for producing a TCH stream, the process comprising: separating, in a first column, an adiponitrile process stream comprising TCH and optionally adiponitrile, to form an adiponitrile stream comprising greater than 5 wt. % adiponitrile and a first TCH stream comprising TCH, and optionally a heavies stream comprising high-boiling components and solid impurities; and optionally purifying the first TCH stream, via one or more columns, to form a purified TCH stream comprising greater than 50 wt. % TCH and optionally less than 1 wt. % impurities and/or less than 1 wt. % decomposition products of high-boiling components, and/or less than 1 wt. % amines. The first column and/or the second column may be operated at a pressure drop less than 25 mmHg. The first column and/or the second column may be a packed column and the packing may comprise high efficiency packing, optionally providing for a pressure drop of less than 0.5 mmHg/theoretical stage. The process may further comprise the step of flashing a crude adiponitrile stream, optionally comprising less than 25 wt. % TCH to form the adiponitrile process stream and a bottoms stream comprising high-boiling components and solid impurities. The purifying may comprise: separating, in a second column, the first TCH stream to form the purified TCH stream and a heavies stream comprising high-boiling components. Residence time may be less than 8 hours. The TCH stream may comprise: TCH, from 0 wt. % to 0.05 wt. % adiponitrile, from 0 wt. % to 0.1 wt. % di(2-cyanoethyl) amine, from 0 wt. % to 0.05 wt. % cyanovaleramide, and from 0 wt. % to 0.05 wt. % tri(2-cyanoethyl) amine.

### BRIEF DESCRIPTION OF THE DRAWINGS

The disclosure is described in detail below with reference to the appended drawings, wherein like numerals designate similar parts.

FIG. 1 depicts a schematic overview of an embodiment of the process for producing an intermediate adiponitrile stream.

FIG. 2 depicts a schematic overview of another embodiment of the process for producing an intermediate adiponitrile stream.

FIG. 3 depicts a schematic overview of another embodiment of the process for producing an intermediate adiponitrile stream.

FIG. 4 depicts a schematic overview of another embodiment of the process for producing an intermediate adiponitrile stream.

FIG. 5 depicts a schematic overview of another embodiment of the process for producing an intermediate adiponitrile stream.

### DETAILED DESCRIPTION

As noted above, some conventional production process streams, e.g., adiponitrile production process streams, contain amounts of desirable components, e.g., tricyanohexane (TCH), e.g., 1,3,6-hexane-tricarbonitrile and/or 1,2,6-hexane-tricarbonitrile. Typically these streams are treated as waste streams, e.g., burned. However, the inventors have found that it is possible to effectively separate and repurpose these streams so as to recover the components present therein. In particular, because TCH is valuable, there is a desire to recover it to yield a (saleable) TCH product.

The inventors have found, however, that the separation of TCH from adiponitrile production process streams is particularly precarious, in some cases due to the make-up of the process stream, e.g., relatively high content of amine impurities and decomposition products (see discussion below). And the literature relating to such separation is sparse. Most separation references relate to treatment of higher TCH content streams—not to process streams comprising relatively small amounts of TCH and the various accompanying impurities.

It has now been discovered that certain separation processes provide for the effective recovery of the lower amounts of TCH in many (adiponitrile) process streams. Because of the effectiveness of the recovery schemes, the TCH is advantageously captured and may be used elsewhere or sold, which results in significant improvements in overall production efficiency. Importantly, when a lower TCH content streams are treated using separation units operating with lower pressure drops (and in some cases low residence times), highly effective separation is achieved. In some cases, the particular treatment of the streams significantly concentrates the TCH, which makes recovery and/or re-use practical and feasible.

Without being bound by theory, it is postulated that amine impurities are particularly difficult to separate from TCH. For example, the presence of components having boiling points close to that of TCH, e.g., CVA, have been found to be problematic in conventional schemes. In many cases, the amine separation is accompanied by high column pressure drop operation, which, in turn, leads to other separation difficulties, e.g., solid degradation.

Further, it is believed that some TCH-containing streams also contain a number of low-boiling and high-boiling impurities. Although conventional methods of separating impurities on the basis of differing boiling points are known, the inventors have found such methods to be unsuccessful in effectively separating TCH from these streams. In particular, it has been discovered that certain high-boiling impurities are prone to decomposition into other impurities, including those with lower or higher boiling points, during conventional separation processes. The decomposition products may have been found to limit the capability of meeting commercially desirable purity of TCH. Conventional TCH recovery processes do not account for this decomposition and, as a result, require additional purification steps, causing lower efficiencies. In particular, the inventors have found that the residence time of the feed streams in the various purification operations affects the decomposition, and that by limiting residence time, e.g., to less than 8 hours in a particular purification operation, optionally at particular pressures and/or temperatures, significant improvements in purification are achieved. Conventional methods of separation and/or purification of TCH provide little or no guidance relating to the effect of these component concentrations, e.g., amine and decomposition product concentrations, on the final TCH yield.

In some cases, the disclosure relates to processes for producing a TCH stream, e.g., from process streams comprising lower concentrations of TCH, which is typically treated as waste. The process comprises the step of separating (in a first column) an adiponitrile process stream to form an adiponitrile stream and a first TCH stream. In some embodiments, the adiponitrile process stream comprises, inter alia, (lower concentrations of) TCH and optionally adiponitrile. The first TCH stream, comprises TCH, e.g., present in higher concentration than in the adiponitrile process stream. The adiponitrile stream comprises adiponitrile, e.g., greater than 10 wt. % adiponitrile. In some cases, the separating step also forms a heavies) stream comprising high-boiling components and solid impurities. The separating takes place in a (first) column, e.g., one or more columns. And, importantly, the first column is operated at a pressure drop less than 25 mmHg. The inventors have found that the low pressure drop operation, optionally in combination with other separation parameters discussed herein, provides for significant process efficiencies, e.g., improvements in solid degradation, while still effectively separating the low TCH content adiponitrile process stream to form the high purity (first) TCH stream. Pressure drop across a column is a well-known measurement and it is discussed at length in many chemical engineering (or chemistry) manuals. The compositions of the aforementioned streams are discussed in more detail below. As noted above, it is believed that the low pressure drop operation works particularly well with the specific adiponitrile process stream due to relatively low content of TCH and the relatively high content of amine impurities and decomposition products.

In some embodiments, the (first) column is operated at a pressure drop less than 25 mmHg, e.g., less than 22 mmHg, less than 20 mmHg, less than 17 mmHg, less than 15 mmHg, less than 13 mmHg, less than 11 mmHg, less than 10 mmHg, less than 8 mmHg, less than 7 mmHg, less than 5 mmHg, or less than 3 mmHg. In terms of ranges, the (first) column may be operated at a pressure drop ranging from 0 mmHg to 25 mmHg, e.g., from 0.5 mmHg to 23 mmHg, from 1 mmHg to 20 mmHg, from 2 mmHg to 15 mmHg, from 1 mmHg to 11 mmHg, from 3 mmHg to 12 mmHg, from 5 mmHg to 11 mmHg, or from 5 mmHg to 7 mmHg. In terms of upper limits, the (first) column may be operated at a pressure drop greater than 0 mmHg, e.g., greater than 0.1 mmHg, greater than 0.5 mmHg, greater than 1 mmHg, greater than 2 mmHg, greater than 3 mmHg, greater than 5 mmHg, or greater than 6 mmHg.

In some cases, the first column (and/or any of the subsequent purification columns) is a packed column, and in some embodiments, the packing comprises high efficiency packing, The high efficiency packing may advantageously provide for a pressure drop per theoretical stages of less than 1.5 mmHg/theoretical stage, e.g., less than 1.0 mmHg, less than 0.9 mmHg, less than 0.75 mmHg, less than 0.6 mmHg, less than 0.5 mmHg, less than 0.45, less than 0.4, less than 0.35, less than 0.3, less than 0.25, less than 0.2, less than 0.1, or less than 0.05 mmHg/theoretical stage. In terms of ranges, the high efficiency packing may provide for a pressure drop per theoretical stages ranging from 0.01 mmHg to 1.5 mmHg, e.g., from 0.01 mmHg to 1.0 mmHg, from 0.05 mmHg to 1.0 mmHg, from 0.1 mmHg to 1.0 mmHg, from 0.2 mmHg to 0.9 mmHg, from 0.3 mmHg to 0.6 mmHg, from 0.01 to 0.45, from 0.05 to 0.4, from 0.1 to 0.35, from 0.15 to 0.35, or from 0.2 to 0.3. The aforementioned operating parameters are applicable to other columns as well.

Also, the high efficiency packing may advantageously provide for a high number of theoretical stages per given volume.

In some cases, the first column (and/or any of the subsequent purification columns) may operate with a short residence time. The residence time of feed streams in the individual separation and/or purification operations of the process is minimized, e.g., less than 8 hours, e.g., less than 7 hours, less than 6 hours, less than 5 hours, or less than 4 hours. The lower residence times (optionally in combination with the lower pressure drop) unexpectedly contributes to the separation/purification efficiencies.

In some embodiments, the processes comprise the optional step of purifying the first TCH stream, via one or more (additional) columns, to form a purified TCH stream, which is a high purity TCH stream, e.g., comprising greater than 50 wt. % TCH. In some cases, the purification step(s) are also carried out in column(s) operating under the conditions disclosed for the first column, e.g., under low pressure drops and low residence times. As an example, the first and second columns may be packed columns and may be operated at a pressure drop less than 25 mmHg. Similar benefits have been found in these purification columns. The resulting purified TCH stream has a low impurity content, e.g., a low decomposition product impurity content.

In some embodiments, the adiponitrile process stream may result from a flashing operation. Stated another way, a general crude adiponitrile stream (comprising TCH and adiponitrile) may first be flashed to form the adiponitrile process stream before being conveyed to the first column. In some cases, the processes comprise the step of flashing a crude adiponitrile stream to form the adiponitrile process stream and a bottoms stream comprising high-boiling components and solid impurities.

### Crude Adiponitrile Stream

As noted, the crude adiponitrile stream and the adiponitrile process stream have specific compositions, which have surprisingly been found to separate efficiently when employing the disclosed processes. In particular, the crude adiponitrile stream may comprise TCH, adiponitrile, high-boiling components, and low boiling components. Conventional separation processes have had difficulty in isolating the lower quantities of TCH and/or adiponitrile. In some embodiments, the crude adiponitrile stream may be one or more process streams of another industrial chemical production process. For example, the feed stream may comprise one or more process streams from different processes or systems, e.g., the production of adiponitrile, acrylonitrile, allyl cyanide, butyronitrile, polyacrylonitrile, polyamides, polyaramids, or combinations thereof. In a specific case, the crude adiponitrile stream may be an adiponitrile process stream, e.g., one or more process streams, purge streams, or flash tails from an adiponitrile production process. In some cases, streams from multiple processes may be combined to form the stream. In conventional process, such TCH-containing (and/or adiponitrile-containing) streams are often treated as waste streams, e.g., vented or burned, and the valuable components are not recovered. By recovering TCH and/or adiponitrile from these streams, as described herein, the TCH may be recovered and used or sold, thus increasing efficiency and profitability.

In some embodiments, the crude adiponitrile process stream comprises less than 70 wt. % TCH, e.g., less than 50 wt. %, less than 35 wt. %, less than 25 wt %, less than 20 wt %, less than 18 wt %, less than 15 wt %, less than 12 wt %, less than 10 wt %, or less than 5 wt %. In terms of ranges, the crude adiponitrile process stream may comprise from 0.1 wt. % to 70 wt. % TCH, e.g., from 0.1 wt. % to 50 wt. %, from 0.1 wt. % to 35 wt. %, from 0.1 wt % to 25 wt %, from 0.5 wt % to 23 wt %, from 0.5 wt % to 20 wt %, from 1 wt % to 15 wt %, from 1.5 wt % to 12 wt %, or from 2 wt % to 11 wt %. In terms of lower limits, the crude adiponitrile process stream may comprise greater than 0.1 wt % TCH, e.g., greater than 0.3 wt %, greater than 0.5 wt %, greater than 0.7 wt %, greater than 1.0 wt %, greater than 1.5 wt %, greater than 2 wt %, or greater than 5 wt %.

In some embodiments, the crude adiponitrile stream comprises higher amounts of TCH. In one embodiment, the feed stream comprises TCH in an amount ranging from 0 wt. % to 90 wt. %, based on the total weight of the feed stream, e.g., from 0 wt. %, to 89 wt. %, from 0 wt. % to 88 wt. %, from 0 wt. % to 85 wt. %, from 0 wt. % to 84 wt. %, from 10 wt. % to 90 wt. %, from 10 wt. %, to 89 wt. %, from 10 wt. % to 88 wt. %, from 10 wt. % to 85 wt. %, from 10 wt. % to 84 wt. %, from 20 wt. % to 90 wt. %, from 20 wt. %, to 89 wt. %, from 20 wt. % to 88 wt. %, from 20 wt. % to 85 wt. %, from 20 wt. % to 84 wt. %, from 30 wt. % to 90 wt. %, from 30 wt. %, to 89 wt. %, from 30 wt. % to 88 wt. %, from 30 wt. % to 85 wt. %, from 30 wt. % to 84 wt. %, from 40 wt. % to 90 wt. %, from 40 wt. %, to 89 wt. %, from 40 wt. % to 88 wt. %, from 40 wt. % to 85 wt. %, from 40 wt. % to 84 wt. %, from 50 wt. % to 90 wt. %, from 50 wt. %, to 89 wt. %, from 50 wt. % to 88 wt. %, from 50 wt. % to 85 wt. %, or from 50 wt. % to 84 wt. %. In terms of upper limits, the crude adiponitrile stream may comprise less than 90 wt. % TCH, e.g., 89 wt. %, less than 88 wt. %, less than 85 wt. %, or less than 84 wt. %, In terms of lower limits, the crude acrylonitrile stream may comprise greater than 0 wt. % TCH, e.g., greater than 10 wt. %, greater than 20 wt. %, greater than 30 wt. %, greater than 40 wt. %, greater than 50 wt %, greater than 60 wt %, or greater than 70 wt %.

The crude adiponitrile process stream may comprise less than 90 wt % adiponitrile, e.g., less than 75 wt. %, less than 50 wt. %, less than 40 wt %, less than 35 wt %, less than 30 wt %, less than wt 20%, less than 18 wt %, less than 15 wt %, less than 12 wt %, less than 10 wt %, or less than 5 wt %. In terms of ranges, the crude adiponitrile process stream may comprise from 0.1 wt. % to 90 wt. % adiponitrile, e.g., from 0.1 wt. % to 75 wt. %, from 0.1 wt % to 40 wt % adiponitrile, from 0.5 wt % to 30 wt %, from 1 wt % to 20 wt %, from 1 wt % to 18 wt %, from 1 wt % to 10 wt %, from 2 wt % to 15 wt %, from 3 wt % to 15 wt %, or from 5 wt % to 15 wt %. In terms of lower limits, the crude adiponitrile process stream may comprise greater than 0.1 wt % adiponitrile, e.g., greater than 0.3 wt %, greater than 0.5 wt %, greater than 0.7 wt %, greater than 1.0 wt %, greater than 1.5 wt %, greater than 2 wt %, or greater than 5 wt %.

In some cases, the crude adiponitrile process stream also comprises low-boiling components. Generally, the low-boiling components are impurities having relatively low boiling points. For example, each of the low-boiling components may have a boiling point of less than 415° C., e.g., less than 410° C., less than 400° C., less than 395° C., or less than 390° C. Examples of low-boiling components that may be present in the crude adiponitrile process stream include various cyanocarbons, e.g., acrylonitrile, propionitrile, hydroxypropionitrile, monocyanoethyl propylamine, succinonitrile, methylglutaronitrile, adiponitrile, 2-cyanocyclopentylidenimine, bis-2-cyanoethyl ether, di(2-cyanoethyl) amine, di-2-cyanoethyl propylamine, cyanovaleramide and combinations thereof. In some cases, the term “lights” refers to components that have lower boiling points, e.g., lower boiling points than adiponitrile or lower boiling points than TCH.

In one embodiment, the crude adiponitrile process stream comprises low-boiling components in an amount ranging from 0 wt. % to 70 wt. %, e.g., from 0 wt. %, to 65 wt. %, from 0 wt. % to 60 wt. %, from 0 wt. % to 55 wt. %, from 0 wt. % to 50 wt. %, from 5 wt. % to 70 wt. %, from 5 wt. %, to 65 wt. %, from 5 wt. % to 60 wt. %, from 5 wt. % to 55 wt. %, from 5 wt. % to 50 wt. %, from 10 wt. % to 70 wt. %, from 10 wt. %, to 65 wt. %, from 10 wt. % to 60 wt. %, from 10 wt. % to 55 wt. %, from 10 wt. % to 50 wt. %, from 12 wt. % to 70 wt. %, from 12 wt. %, to 65 wt. %, from 12 wt. % to 60 wt. %, from 12 wt. % to 55 wt. %, from 1 wt % to 20 wt %, from 2 wt % to 15 wt %, from 3 wt % to 15 wt %, from 1 wt % to 10 wt %, from 12 wt. % to 50 wt. %, from 15 wt. % to 70 wt. %, from 15 wt. %, to 65 wt. %, from 15 wt. % to 60 wt. %, from 15 wt. % to 55 wt. %, or from 15 wt. % to 50 wt. %. In terms of upper limits, the crude adiponitrile process stream may comprise less than 70 wt. % low-boiling components, e.g., less than 65 wt. %, less than 60 wt. %, less than 55 wt. %, less than 50 wt. %, less than 20 wt %, less than 15 wt %, or less than 15 wt %. In terms of lower limits, the crude adiponitrile process stream may comprise greater than 0 wt. %, low-boiling components, e.g., greater than 1 wt %, greater than 2 wt %, greater than 3 wt %, greater than 5 wt. %, greater than 10 wt. %, greater than 12 wt. %, or greater than 15 wt. %.

The crude adiponitrile stream also comprises high-boiling components. Generally, the high-boiling components are impurities having relatively high boiling points. For example, each of the high-boiling components may have a boiling point of greater than 395° C., e.g., greater than 400° C., greater than 405° C., greater than 408° C., greater than 410° C., or greater than 415° C. Examples of high-boiling components that may be present in the crude adiponitrile stream include isomeric tricyanohexane, tri(2-cyanoethyl)amine, and combinations thereof. In some cases, the term “heavies” refers to components that have higher boiling points, e.g., higher boiling points than adiponitrile or higher boiling points than TCH.

In one embodiment, the crude adiponitrile process stream comprises high-boiling components in an amount ranging from 0 wt. % to 50 wt. %, e.g., from 0 wt. % to 40 wt. %, from 0 wt. % to 35 wt. %, from 0 wt. % to 25 wt. %, from 0 wt. % to 20 wt. %, from 0.5 wt. % to 50 wt. %, from 0.5 wt. % to 40 wt. %, from 0.5 wt. % to 35 wt. %, from 0.5 wt. % to 25 wt. %, from 0.5 wt. % to 20 wt. %, from 1 wt. % to 50 wt. %, from 1 wt. % to 40 wt. %, from 1 wt. % to 35 wt. %, from 1 wt. % to 25 wt. %, from 1 wt. % to 20 wt. %, from 2 wt. % to 50 wt. %, from 2 wt. % to 40 wt. %, from 2 wt. % to 35 wt. %, from 2 wt. % to 25 wt. %, from 2 wt. % to 20 wt. %, from 3 wt. % to 50 wt. %, from 3 wt. % to 40 wt. %, from 3 wt. % to 35 wt. %, from 3 wt. % to 25 wt. %, from 3 wt. % to 20 wt. %, from 5 wt % to 15 wt %, from 5 wt. % to 50 wt. %, from 5 wt. % to 40 wt. %, from 5 wt. % to 35 wt. %, from 5 wt. % to 25 wt. %, or from 5 wt. % to 20 wt. %. In terms of upper limits, the crude adiponitrile process stream may comprise less than 50 wt. % high-boiling components, e.g., less than 40 wt. %, less than 35 wt. %, less than 30 wt. %, less than 25 wt. % or less than 20 wt. %. In terms of lower limits, the crude adiponitrile process stream may comprise greater than 0 wt. %, e.g., greater than 0.5 wt. %, greater than 1 wt. %, greater than 2 wt. %, greater than 3 wt. %, or greater than 5 wt. %.

In one embodiment, the crude adiponitrile process stream comprises low-boiling components (lights) in an amount ranging from 0 wt. % to 70 wt. %, e.g., from 0 wt. %, to 65 wt. %, from 0 wt. % to 60 wt. %, from 0 wt. % to 55 wt. %, from 0 wt. % to 50 wt. %, from 5 wt. % to 70 wt. %, from 5 wt. %, to 65 wt. %, from 5 wt. % to 60 wt. %, from 5 wt. % to 55 wt. %, from 5 wt. % to 50 wt. %, from 10 wt. % to 70 wt. %, from 10 wt. %, to 65 wt. %, from 10 wt. % to 60 wt. %, from 10 wt. % to 55 wt. %, from 10 wt. % to 50 wt. %, from 12 wt. % to 70 wt. %, from 12 wt. %, to 65 wt. %, from 12 wt. % to 60 wt. %, from 12 wt. % to 55 wt. %, from 1 wt % to 20 wt %, from 2 wt % to 15 wt %, from 3 wt % to 15 wt %, from 1 wt % to 10 wt %, from 12 wt. % to 50 wt. %, from 15 wt. % to 70 wt. %, from 15 wt. %, to 65 wt. %, from 15 wt. % to 60 wt. %, from 15 wt. % to 55 wt. %, or from 15 wt. % to 50 wt. %. In terms of upper limits, the crude adiponitrile process stream may comprise less than 70 wt. % low-boiling components, e.g., less than 65 wt. %, less than 60 wt. %, less than 55 wt. %, less than 50 wt. %, less than 20 wt %, less than 15 wt %, or less than 15 wt %. In terms of lower limits, the crude adiponitrile process stream may comprise greater than 0 wt. %, low-boiling components, e.g., greater than 1 wt %, greater than 2 wt %, greater than 3 wt %, greater than 5 wt. %, greater than 10 wt. %, greater than 12 wt. %, or greater than 15 wt. %.

In one embodiment, the crude adiponitrile stream comprises heavies in an amount ranging from 0 wt. % to 20 wt. %, e.g., from 0 wt. % to 15 wt. %, from 0 wt. % to 10 wt. %, from 0 wt. % to 8 wt. %, from 0 wt. % to 5 wt. %, from 0.5 wt. % to 20 wt. %, from 0.5 wt. % to 15 wt. %, from 0.5 wt. % to 10 wt. %, from 0.5 wt. % to 8 wt. %, from 0.5 wt. % to 5 wt. %, from 1 wt. % to 20 wt. %, from 1 wt. % to 15 wt. %, from 1 wt. % to 10 wt. %, from 1 wt. % to 8 wt. %, from 1 wt. % to 5 wt. %, from 1.5 wt. % to 20 wt. %, from 1.5 wt. % to 15 wt. %, from 1.5 wt. % to 10 wt. %, from 1.5 wt. % to 8 wt. %, from 1.5 wt. % to 5 wt. %, from 2 wt. % to 20 wt. %, from 2 wt. % to 15 wt. %, from 2 wt. % to 10 wt. %, from 2 wt. % to 8 wt. %, from 2 wt. % to 5 wt. %, from 2.5 wt. % to 20 wt. %, from 2.5 wt. % to 15 wt. %, from 2.5 wt. % to 10 wt. %, from 2.5 wt. % to 8 wt. %, or from 2.5 wt. % to 5 wt. %. In terms of upper limits, the crude adiponitrile stream may comprise less than 20 wt. % heavies, e.g., less than 15 wt. %, less than 10 wt. %, less than 8 wt. %, or less than 5 wt. %. In terms of lower limits, the crude adiponitrile stream may comprise greater than 0 wt. % heavies, e.g., greater than 0.5 wt. %, greater than 1 wt. %, greater than 1.5 wt. %, greater than 2 wt. %, or greater than 2.5 wt. %.

In some embodiments, the crude adiponitrile stream may also comprise solid impurities. These impurities may include various organic impurities that are solid under the temperature and pressure conditions. For example, the solid impurities may include solid cyanocarbon compounds. In one embodiment, the crude adiponitrile stream comprises solid impurities in an amount ranging from 0 wt. % to 25 wt. %, e.g., from 0 wt. % to 20 wt. %, from 0 wt. % to 15 wt. %, or from 0 wt. % to 10 wt. %. In terms of upper limits, the crude adiponitrile stream may comprise less than 25 wt. %, e.g., less than 20 wt. %, less than 15 wt. %, or less than 10 wt. %.

In some embodiments, the crude adiponitrile process stream comprises nitriles (generally, e.g., high boiling point and/or low boiling point nitriles). In one embodiment, the crude adiponitrile process stream comprises nitriles in an amount ranging from 0 wt. % to 90 wt. %, based on the total weight of the feed stream, e.g., from 0 wt. %, to 89 wt. %, from 0 wt. % to 88 wt. %, from 0 wt. % to 85 wt. %, from 0 wt. % to 84 wt. %, from 10 wt. % to 90 wt. %, from 10 wt. %, to 89 wt. %, from 10 wt. % to 88 wt. %, from 10 wt. % to 85 wt. %, from 10 wt. % to 84 wt. %, from 20 wt. % to 90 wt. %, from 20 wt. %, to 89 wt. %, from 20 wt. % to 88 wt. %, from 20 wt. % to 85 wt. %, from 20 wt. % to 84 wt. %, from 30 wt. % to 90 wt. %, from 30 wt. %, to 89 wt. %, from 30 wt. % to 88 wt. %, from 30 wt. % to 85 wt. %, from 30 wt. % to 84 wt. %, from 40 wt. % to 90 wt. %, from 40 wt. %, to 89 wt. %, from 40 wt. % to 88 wt. %, from 40 wt. % to 85 wt. %, from 40 wt. % to 84 wt. %, from 50 wt. % to 90 wt. %, from 50 wt. %, to 89 wt. %, from 50 wt. % to 88 wt. %, from 50 wt. % to 85 wt. %, or from 50 wt. % to 84 wt. %. In terms of upper limits, the crude adiponitrile process stream may comprise less than 90 wt. % nitriles, e.g., 89 wt. %, less than 88 wt. %, less than 85 wt. %, or less than 84 wt. %, In terms of lower limits, the crude adiponitrile process stream may comprise greater than 0 wt. % nitriles, e.g., greater than 10 wt. %, greater than 20 wt. %, greater than 30 wt. %, greater than 40 wt. %, or greater than 50.

### Flashing and Adiponitrile Process Stream

As noted above, the crude adiponitrile stream is separated in a flashing step to form the adiponitrile process stream (an overhead stream) comprising adiponitrile and low-boiling components (lights) and (optionally lower amounts of) high-boiling components (heavies) and a first bottoms stream comprising high-boiling components and solid impurities. The flashing step, in some cases, removes a significant portion (if not all) of the heavies and/or the solid impurities present in the crude adiponitrile stream. The inventors have found that removal of the heavies prior to further processing beneficially reduces the decomposition of the high-boiling components and thereby improves the efficiency of the total purification process. Without this initial removal of heavies, additional non-TCH components are formed, which must then be separated, creating additional operations and uncertainties. Furthermore, the inventors have also found that early removal of the heavies and the solid impurities reduces fouling of columns, which improves downstream efficiency and eliminates or reduces the need for subsequent separation operations. The residence time of the feed stream in the flashing may be a short residence time as discussed herein.

In some embodiments, the first separating step includes separation in a flasher, e.g., a flash evaporator. In these embodiments, the crude adiponitrile stream is evaporated and separated into an overhead stream e.g., the adiponitrile process stream, and the first bottoms stream. Various flashers are known to those of ordinary skill in the art, and any suitable flasher may be employed as long as the separation described herein is achieved. In some embodiments, the separation in the flasher may be caused by reducing the pressure, e.g., an adiabatic flash, without heating the feed stream. In other embodiments, the separation in the flasher may be caused by raising the temperature of the feed stream without changing the pressure. In still other embodiments, the separation in the flasher may be caused by reducing the pressure while heating the feed stream. In some embodiments, the first separating step is achieved via a wiped film evaporator (WFE).

In some embodiments, the flashing step includes separating the crude adiponitrile stream in a flash evaporator at reduced pressure, e.g., under a vacuum. In some embodiments, the pressure in the flash evaporator is reduced to less than 25 torr, e.g., less than 20 torr, less than 10 torr, less than 5 torr, or less than 1 torr. In some embodiments, the flash vessel of the flashing step is kept at a constant temperature. In some embodiments, the temperature of the flash vessel may be from 175° C. to 235° C., e.g., from 180° C. to 230° C., from 185° C. to 225° C., or from 190° C. to 220° C. The first bottoms stream comprises high-boiling components (heavies). Examples of heavies that may be present in the first bottoms stream include isomeric tricyanohexane, tri(2-cyanoethyl)amine, and combinations thereof. In one embodiment, the first separation step occurs in a flasher, and the first bottoms stream comprises isomeric tricyanohexane and tri(2-cyanoethyl)amine. The first bottoms stream also may comprise solid impurities. In one embodiment, the flashing step removes all (substantially all) of the solid impurities from the crude adiponitrile stream. Said another way, in this embodiment, the flash overhead stream comprises effectively 0 wt. % solid impurities. In other embodiments, the flashing step may remove less than 100% of the solid impurities, e.g., less than 99.9%, less than 99%, or less than 98%.

In some embodiments, the adiponitrile process stream comprises less than 99 wt. % TCH, e.g., less than 97 wt %, less than 90 wt %, less than 80 wt %, less than 70 wt. % TCH, e.g., less than 50 wt. %, less than 35 wt. %, less than 25 wt. %, less than 20 wt. %, less than 18 wt. %, less than 15 wt. %, less than 12 wt. %, less than 10 wt. %, or less than 5 wt. %. In terms of ranges, the crude adiponitrile stream may comprise from 0.1 wt % to 99 wt % TCH, e.g., from 50 wt % to 99 wt %, from 75 wt % to 98 wt %, from 85 wt % 98 wt %, from 90 wt % to 97 wt %, from 0.1 wt. % to 25 wt. %, from 0.1 wt. % to 70 wt. %, from 0.1 wt. % to 50 wt. %, from 0.1 wt. % to 35 wt. %, from 0.5 wt. % to 23 wt. %, from 0.5 wt. % to 20 wt. %, from 1 wt. % to 15 wt. %, from 1.5 wt. % to 12 wt. %, or from 2 wt. % to 11 wt. %. In terms of lower limits, the crude adiponitrile stream may comprise greater than 0.1 wt. % TCH, e.g., greater than 0.3 wt. %, greater than 0.5 wt. %, greater than 0.7 wt. %, greater than 1.0 wt. %, greater than 1.5 wt. %, greater than 2 wt. %, greater than 5 wt. %, greater than 25 wt %, greater than 50 wt %, greater than 75 wt %, greater than 85 wt %, greater than 85 wt %, or greater than 90 wt %.

The adiponitrile process stream may comprise less than 90 wt % adiponitrile, e.g., less than 75 wt %, less than 50 wt %, less than 40 wt %, less than 35 wt %, less than 30 wt %, less than wt 20%, less than 18 wt %, less than 15 wt %, less than 12 wt %, less than 10 wt %, less than 5 wt %, less than 4 wt %, less than 3 wt %, or less than 2 wt %. In terms of ranges, the adiponitrile process stream may comprise from 0.1 wt % to 90 wt % adiponitrile, e.g., from 0.1 wt % to 75 wt %, from 0.1 wt % to 40 wt %, from 0.1 wt % to 10 wt %, from 0.1 wt % to 5 wt %, from 0.5 wt % to 5 wt %, from 0.5 wt % to 3 wt %, from 0.5 wt % to 30 wt %, from 1 wt % to 20 wt %, from 2 wt % to 20 wt %, from 5 wt % to 18 wt %, or from 5 wt % to 15 wt %. In terms of lower limits, the adiponitrile process stream may comprise greater than 0.1 wt % adiponitrile, e.g., greater than 0.3 wt %, greater than 0.5 wt %, greater than 0.7 wt %, greater than 1.0 wt %, greater than 1.5 wt %, greater than 2 wt %, or greater than 5 wt %.

In one embodiment, the adiponitrile process stream comprises lights in an amount ranging from 0 wt. % to 70 wt. %, e.g., from 0.1 wt % to 30 wt %, from 0.1 wt % to 50 wt %, from 0 wt. % to 25 wt. %, from 0 wt. %, to 20 wt. %, from 0 wt. % to 15 wt. %, from 0 wt. % to 10 wt. %, from 1 wt. % to 30 wt. %, from 1 wt. % to 25 wt. %, from 1 wt. %, to 20 wt. %, from 1 wt. % to 15 wt. %, from 1 wt. % to 10 wt. %, from 2 wt. % to 30 wt. %, from 2 wt. % to 25 wt. %, from 2 wt. %, to 20 wt. %, from 2 wt. % to 15 wt. %, from 2 wt. % to 10 wt. %, from 3 wt. % to 30 wt. %, from 3 wt. % to 25 wt. %, from 3 wt. %, to 20 wt. %, from 0.1 wt. %, to 10 wt. %, from 0.1 wt. %, to 5 wt. %, from 0.3 wt. %, to 3 wt. %, from 0.5 wt. %, to 2 wt. %, from 1 wt. %, to 3 wt. %, from 3 wt. % to 15 wt. %, from 3 wt. % to 10 wt. %, from 4 wt. % to 30 wt. %, from 4 wt. % to 25 wt. %, from 4 wt. %, to 20 wt. %, from 4 wt. % to 15 wt. %, from 4 wt. % to 10 wt. %, from 5 wt. % to 30 wt. %, from 5 wt. % to 25 wt. %, from 5 wt. %, to 20 wt. %, from 5 wt. % to 15 wt. %, or from 5 wt. % to 10 wt. %. In terms of upper limits, the adiponitrile process stream may comprise less than 70 wt. % lights, e.g., less than 50 wt %, less than 30 wt %, less than 25 wt. %, less than 20 wt. %, less than 15 wt. %, less than 10 wt. %, less than 5 wt %, less than 3 wt %, or less than 2 wt %. In terms of lower limits, the adiponitrile process stream may comprise greater than 0 wt. % lights, e.g., greater than 0.1 wt %, greater than 0.3 wt %, greater than 0.5 wt %, greater than 1 wt. %, greater than 2 wt. %, greater than 3 wt. %, greater than 4 wt. %, or greater than 5 wt. %.

In one embodiment, the adiponitrile process stream comprises heavies in an amount ranging from 0 wt. % to 20 wt. %, e.g., from 0 wt. % to 15 wt. %, from 0 wt. % to 10 wt. %, from 0 wt. % to 8 wt. %, from 0 wt. % to 5 wt. %, from 0.5 wt. % to 20 wt. %, from 0.5 wt. % to 15 wt. %, from 0.5 wt. % to 10 wt. %, from 0.5 wt. % to 8 wt. %, from 0.5 wt. % to 5 wt. %, from 1 wt. % to 20 wt. %, from 1 wt. % to 15 wt. %, from 1 wt. % to 10 wt. %, from 1 wt. % to 8 wt. %, from 1 wt. % to 5 wt. %, from 1.5 wt. % to 20 wt. %, from 1.5 wt. % to 15 wt. %, from 1.5 wt. % to 10 wt. %, from 1.5 wt. % to 8 wt. %, from 1.5 wt. % to 5 wt. %, from 2 wt. % to 20 wt. %, from 2 wt. % to 15 wt. %, from 2 wt. % to 10 wt. %, from 2 wt. % to 8 wt. %, from 2 wt. % to 5 wt. %, from 2.5 wt. % to 20 wt. %, from 2.5 wt. % to 15 wt. %, from 2.5 wt. % to 10 wt. %, from 2.5 wt. % to 8 wt. %, or from 2.5 wt. % to 5 wt. %. In terms of upper limits, the adiponitrile process stream may comprise less than 20 wt. % heavies, e.g., less than 15 wt. %, less than 10 wt. %, less than 8 wt. %, or less than 5 wt. %. In terms of lower limits, the adiponitrile process stream may comprise greater than 0 wt. % heavies, e.g., greater than 0.5 wt. %, greater than 1 wt. %, greater than 1.5 wt. %, greater than 2 wt. %, or greater than 2.5 wt. %.

In some cases, the flashing step removes a significant portion of the heavies from the crude adiponitrile stream. Said another, the adiponitrile process stream comprises low amounts, if any, of the heavies initially present in the feed stream. In some embodiments, the adiponitrile process stream comprises less than 70% of the heavies present in the feed stream, e.g., less than 65%, less than 60%, less than 55%, or less than 50%.

### Separating and First TCH Stream

As noted above, the adiponitrile process stream is separated in a separating step to form the first TCH stream, the adiponitrile stream comprising adiponitrile and lights (low-boiling components), and a heavies stream comprising heavies (high-boiling components). The separating step, in some cases, removes a significant portion (if not all) of the low-boiling components and high-boiling components present in the adiponitrile process stream. In some cases, the separating step comprises one or more columns, e.g., two columns. In some embodiments, the separating step comprise two columns and the first distillation column forms a lights stream as an overhead stream (comprising adiponitrile) and a second bottoms stream. The second bottoms stream is then separated in a second distillation column to form the heavies stream as a third bottoms stream and the TCH stream as a third overhead stream.

The various separating steps discussed herein may include separation of the adiponitrile process stream in one or more distillation columns and/or in one or more flash evaporators. The structure of the one or more distillation columns may vary widely. Various distillation columns are known to those of ordinary skill in the art, and any suitable column may be employed in the second separation step as long as the separation described herein is achieved. For example, the distillation column may comprise any suitable separation device or combination of separation devices. For example, the distillation column may comprise a column, e.g., a standard distillation column, a packed column, an extractive distillation column and/or an azeotropic distillation column. Similarly, as noted above, various flashers are known to those of ordinary skill in the art, and any suitable flasher may be employed in the second separation step as long as the separation described herein is achieved. For example, the flasher may comprise an adiabatic flash evapaorator, a heated flash evaporator, or a wipe film evaporator, or combinations thereof.

Embodiments of the separating step may include any combination of one or more distillation columns and/or one or more flashers, as long as the aforementioned streams are formed.

In one embodiment, for example, the separating step comprises separating the adiponitrile process stream in two consecutive distillation columns. In this embodiment, the first overhead lights stream is separated in a first distillation column. A second overhead lights stream is collected from the overhead (e.g., the column top and/or a relatively high side draw) of the first distillation column, and a second bottom (intermediate) heavies stream is collected from the bottom (e.g., the column bottom and/or a relatively low side draw) of the first distillation column. At least a portion of the second bottom (intermediate) heavies stream is then separated in a second distillation column. A third bottom heavies stream is collected from the bottom (e.g., the column bottom and/or a relatively low side draw) of the second distillation column. The TCH stream is collected from the overhead (e.g., column top and/or a relatively high side draw) of the second distillation column, e.g., as a third overhead lights stream.

In another embodiment, the separating step comprises separating the adiponitrile process stream in a three distillation columns. In this embodiment, this stream is separated in a first distillation column. A second overhead lights stream is collected from the overhead (e.g., the column top and/or a relatively high side draw) of the first distillation column, and a second bottom heavies stream is collected from the bottom (e.g., the column bottom and/or a relatively low side draw) of the first distillation column. At least a portion of the second bottom heavies stream is then separated in a second distillation column. A third overhead lights stream is collected from the overhead (e.g., the column top and/or a relatively high side draw) of the second distillation column, and third bottom heavies stream is collected from the bottom (e.g., the column bottom and/or a relatively low side draw) of the second distillation column. At least a portion of the third overhead lights stream is then separated in a third distillation column. A fourth bottom heavies stream is collected from the bottom (e.g., the column bottom and/or a relatively low side draw) of the third distillation column, and the TCH stream is collected from the top (e.g., the column top and/or a relatively high side draw) of the third distillation column, e.g., as a fourth overhead lights stream.

In another embodiment, the separating step comprises separating the adiponitrile process stream in a two distillation columns and an evaporator (e.g., flasher, WFE, or falling film evaporator). In this embodiment, the first overhead lights stream is separated in a first distillation column. A second overhead lights stream is collected from the overhead (e.g., the column top and/or a relatively high side draw) of the first distillation column, and a second bottom heavies stream is collected from the bottom (e.g., the column bottom and/or a relatively low side draw) of the first distillation column. At least a portion of the second bottom heavies stream is then separated in a second distillation column. A third overhead lights stream is collected from the overhead (e.g., the column top and/or a relatively high side draw) of the second distillation column, and third bottom heavies stream is collected from the bottom (e.g., the column bottom and/or a relatively low side draw) of the second distillation column. At least a portion of the third overhead lights stream is then separated in an evaporator. A fourth overhead lights stream is collected from the top of the evaporator, and the TCH stream is collected from the bottom of the evaporator, e.g., as a fourth bottom heavies stream.

As noted above, low pressure drop column operation has been found to be unexpectedly effective. For example, low pressure drop operation, optionally in combination with other separation parameters discussed herein, provides for significant process efficiencies, e.g., improvements in solid degradation, while still effectively separating the low TCH content adiponitrile process stream to form the high purity (first) TCH stream.

### TCH Stream

As a result of the disclosed operation parameters, in some embodiments, the (first) TCH stream may comprise greater than 1 wt % TCH, e.g., greater than 5 wt %, greater than 10 wt %, greater than 20 wt %, greater than 25 wt %, greater than 30 wt %, greater than 35 wt %, greater than 50 wt %, greater than 75 wt %, greater than 85 wt %, greater than 90 wt %, greater than 93%, or greater than 95 wt %. In terms of ranges, the first TCH stream may comprise from 1 wt % to 99.9 wt % TCH, e.g., from 25 wt % to 99.9 wt %, from 50 wt % to 99.9 wt %, from 75 wt % to 99.9 wt %, from 90 wt % to 99.9 wt %, from 85 wt % to 99.5 wt %, from 5 wt % to 99 wt %, from 50 wt % to 99 wt %, from 5 wt % to 95 wt %, from 25 wt % to 90 wt %, from 45 wt % to 90 wt %, or from 50 wt % to 85 wt %. In terms of upper limits, the first TCH stream comprises less than 99.9 wt % TCH, e.g., less than 99 wt %, less than 99.5 wt %, less than 95 wt %, less than wt 90%, less than 85 wt %, less than 80 wt %, less than 75 wt %, or less than 65 wt %.

In some embodiments, the (first) TCH stream comprises TCH in higher amounts ranging from 90 wt. % to 100 wt. %, e.g., from 90 wt. % to 99.9 wt. %, from 90 wt. % to 99 wt. %, from 90 wt. % to 98 wt. %, from 92.5 wt. % to 100 wt. %, from 92.5 wt. % to 99.9 wt. %, from 92.5 wt. % to 99 wt. %, from 92.5 to 98 wt. %, from 95 wt. % to 100 wt. %, from 95 wt. % to 99.9 wt. %, from 95 wt. % to 99 wt. %, from 95 to 98 wt. %, from 97.5 wt. % to 100 wt. %, from 97.5 wt. % to 99.9 wt. %, from 97.5 to 99 wt. %, or from 97.5 to 98 wt. %. In terms of upper limits, the TCH stream may comprise less than 100 wt. % TCH, e.g., less than 99.9 wt. % less than 99 wt. %, or less than 98 wt. %. In terms of lower limits, the TCH stream may comprise greater than 90 wt. %, e.g., greater than 92.5 wt. %, greater than 95 wt. %, or greater than 97.5 wt. %. Conventional processes have been unable to achieve such high TCH purity levels.

In one embodiment, the TCH stream comprises impurities, e.g., heavies and/or lights, in an amount ranging from 0 wt. % to 10 wt. %, e.g., from 0 wt. % to 7.5 wt. %, from 0 wt. % to 5 wt. %, from 0 wt. % to 2.5 wt. %, from 0.1 wt. % to 10 wt. %, from 0.1 wt. % to 7.5 wt. %, from 0.1 wt. % to 5 wt. %, from 0.1 wt. % to 2.5 wt. %, 0.1 wt. % to 1.5 wt. %, 0.2 wt. % to 1.2 wt. %, 0.3 wt. % to 1.5 wt. %, 0.5 wt. % to 1.0 wt. %, from 1 wt. % to 10 wt. %, from 1 wt. % to 7.5 wt. %, from 1 wt. % to 5 wt. %, from 1 wt. % to 2.5 wt. %, from 2 wt. % to 10 wt. %, from 2 wt. % to 7.5 wt. %, from 2 wt. % to 5 wt. %, or from 2 wt. % to 2.5 wt. %. In terms of upper limits, the TCH stream may comprise less than 10 wt. % impurities, e.g., less than 7.5 wt. %, less than 5 wt. %, less than 2.5 wt. %, less than 1.5 wt. %, less than 1.2 wt. %, or less than 1.0 wt. %. In terms of lower limits, the TCH stream may comprise greater than 0 wt. % impurities, e.g., greater than 0.1 wt. %, greater than 1 wt. %, or greater than 2 wt. %. The TCH stream may comprise amines and/or nitriles in these amounts. In some cases, the use of lower pressures in the separation surprisingly provides for improved separation of components having boiling points close to that of TCH, e.g., CVA. These ranges and limits apply to heavies and lights individually or combined.

In one embodiment, the first TCH stream comprises from 0 wt. % to 0.05 wt. % adiponitrile, from 0 wt. % to 0.1 wt. % di(2-cyanoethyl) amine, from 0 wt. % to 0.05 wt. % cyanovaleramide, and from 0 wt. % to 0.05 wt. % tri(2-cyanoethyl) amine.

The (first) TCH stream may comprise less than 25 wt. % adiponitrile, e.g., less than 23 wt. %, less than 20 wt. %, less than 18 wt. %, less than 15 wt. %, less than 12 wt. %, less than 10 wt. %, less than 8 wt. %, less than 5 wt. %, less than 3 wt. %, less than 1 wt. %, less than 0.05 wt. %, or less than 0.03 wt. %. In terms of ranges, the (first) TCH stream may comprise from 0.001 wt. % to 25 wt. % adiponitrile, e.g., from 0.05 wt. % to 5 wt. %, from 0.1 wt. % to 25 wt. %, from 0.5 wt. % to 22 wt. %, from 1 wt. % to 20 wt. %, from 2 wt. % to 20 wt. %, or from 5 wt. % to 18 wt. %. In terms of lower limits, the (first) TCH stream may comprise greater than 0.001 wt. % adiponitrile, e.g., greater than 0.01 wt %, greater than 0.01 wt. %, greater than 0.5 wt. %, greater than 1.0 wt. %, greater than 2.0 wt. %, greater than 5.0 wt. %, greater than 10 wt. %, or greater than 15 wt. %.

In one embodiment, the TCH stream comprises from 0 wt. % to 0.05 wt. % adiponitrile, from 0 wt. % to 0.1 wt. % di(2-cyanoethyl) amine, from 0 wt. % to 0.05 wt. % cyanovaleramide, and from 0 wt. % to 0.05 wt. % tri(2-cyanoethyl) amine.

### Adiponitrile Stream

In some embodiments, the adiponitrile stream may comprise greater than 1 wt % TCH, e.g., greater than 5 wt %, greater than 10 wt %, greater than 20 wt %, greater than 25 wt %, greater than 30 wt %, greater than 35 wt %, greater than 50 wt %, greater than 60 wt %, or greater than 70 wt %. In terms of ranges, the adiponitrile stream may comprise from 1 wt % to 95 wt % TCH, from 5 wt % to 95 wt %, from 20 wt % to 95 wt %, from 30 wt % to 95 wt %, from 45 wt % to 80 wt %, from 50 wt % to 95 wt %, from 60 wt % to 90 wt %, from 70 wt % to 90 wt %, from 25 wt % to 75 wt %, from 30 wt % to 70 wt %, or from 40 wt % to 60 wt %. In terms of lower limits, the adiponitrile stream comprises less than 95 wt % TCH, e.g., less than wt 90%, less than 85 wt %, less than 80 wt %, less than 75 wt %, less than 65 wt %, or less than 60 wt %.

In some embodiments, the adiponitrile stream may comprise greater than 1 wt % adiponitrile, e.g., greater than 5 wt %, greater than 6 wt %, greater than 10 wt %, greater than 20 wt %, greater than 25 wt %, greater than 30 wt %, greater than 35 wt %, or greater than 50 wt %. In terms of ranges, the adiponitrile stream may comprise from 1 wt % to 95 wt % adiponitrile, from 5 wt % to 95 wt %, from 7 wt % to 75 wt %, from 5 wt % to 35 wt %, from 6 wt % to 30 wt %, from 25 wt % to 75 wt %, from 30 wt % to 70 wt %, or from 40 wt % to 60 wt %. In terms of lower limits, the adiponitrile stream comprises less than 95 wt % TCH, e.g., less than wt 90%, less than 85 wt %, less than 80 wt %, less than 75 wt %, less than 65 wt %, less than 60 wt %, or less than 30 wt %.

The adiponitrile stream may comprise less than 70 wt % lights, e.g., less than 50 wt %, less than 35 wt %, less than 25 wt %, less than 20 wt %, less than 15 wt %, less than 12 wt %, or less than 10 wt %. In terms of ranges, the adiponitrile stream may comprise from 0.1 wt % to 70 wt % lights, e.g., from 0.1 wt % to 50 wt %, from 0.1 wt % to 25 wt %, from 0.5 wt % to 25 wt %, from 10 wt % to 25 wt %, from 1 wt % to 20 wt %, from 2 wt % to 18 wt %, from 2 wt % to 15 wt %, or from 2 wt % to 10 wt %. In terms of lower limits, the adiponitrile stream may comprise greater than 0.1 wt % lights, e.g., greater than 0.3 wt %, greater than 0.5 wt %, greater than 0.7 wt %, greater than 1.0 wt %, greater than 1.5 wt %, greater than 2 wt %, or greater than 5 wt %. As noted above, in some cases, the term “lights” refers to components that have lower boiling points, e.g., lower boiling points than adiponitrile or lower boiling points than TCH.

The adiponitrile stream comprises high-boiling components (heavies). In one embodiment, the adiponitrile stream comprises high-boiling components in an amount ranging from 0.1 wt % to 50 wt %, e.g., from 0.1 wt. % to 20 wt. %, from 0.1 wt. % to 10 wt. %, from 0.5 wt. % to 10 wt. %, from 0.5 wt. % to 5 wt. %, from 1 wt. % to 3 wt. %, from 5 wt. % to 50 wt. %, e.g., from 5 wt. % to 45 wt. %, from 5 wt. % to 40 wt. %, from 5 wt. % to 35 wt. %, from 5 wt. % to 30 wt. %, from 8 wt. % to 50 wt. %, from 8 wt. % to 45 wt. %, from 8 wt. % to 40 wt. %, from 8 wt. % to 35 wt. %, from 8 wt. % to 30 wt. %, from 10 wt. % to 50 wt. %, from 10 wt. % to 45 wt. %, from 10 wt. % to 40 wt. %, from 10 wt. % to 35 wt. %, from 10 wt. % to 30 wt. %, from 12 wt. % to 50 wt. %, from 12 wt. % to 45 wt. %, from 12 wt. % to 40 wt. %, from 12 wt. % to 35 wt. %, from 12 wt. % to 30 wt. %, from 15 wt. % to 50 wt. %, from 15 wt. % to 45 wt. %, from 15 wt. % to 40 wt. %, from 15 wt. % to 35 wt. %, or from 15 wt. % to 30 wt. %. In terms of upper limits, the adiponitrile stream may comprise less than 50 wt. % high-boiling components, e.g., less than 45 wt. %, less than 40 wt. %, less than 35 wt. %, less than 30 wt. %, less than 20 wt. %, less than 10 wt. %, less than 5 wt. %, or less than 3 wt. %. In terms of lower limits, the adiponitrile stream may comprise greater than 0.1 wt. % high-boiling components, e.g., greater than 0.5 wt %, greater than 1 wt. %, greater than 5 wt. %, greater than 8 wt. %, greater than 10 wt. %, greater than 12 wt. %, or greater than 15 wt. %.

In some cases, the separation may be achieved in a two column system. The first column yields the adiponitrile stream and an intermediate bottoms stream, which is fed to the second column. The intermediate bottoms stream may comprise high amounts of TCH and may then be further separated, e.g., in one or more additional columns. For example, the intermediate bottoms stream, in some embodiments, comprises TCH in high amounts ranging from 90 wt. % to 100 wt. %, e.g., from 90 wt. % to 99.9 wt. %, from 90 wt. % to 99 wt. %, from 90 wt. % to 98 wt. %, from 92.5 wt. % to 100 wt. %, from 92.5 wt. % to 99.9 wt. %, from 92.5 wt. % to 99 wt. %, from 92.5 to 98 wt. %, from 95 wt. % to 100 wt. %, from 95 wt. % to 99.9 wt. %, from 95 wt. % to 99 wt. %, from 95 to 98 wt. %, from 97.5 wt. % to 100 wt. %, from 97.5 wt. % to 99.9 wt. %, from 97.5 to 99 wt. %, or from 97.5 to 98 wt. %. In terms of upper limits, the intermediate bottoms stream may comprise less than 100 wt. % TCH, e.g., less than 99.9 wt. % less than 99 wt. %, or less than 98 wt. %. In terms of lower limits, the intermediate bottoms stream may comprise greater than 90 wt. %, e.g., greater than 92.5 wt. %, greater than 95 wt. %, or greater than 97.5 wt. %.

The intermediate bottoms stream may further comprise small amounts of adiponitrile and lights (amounts similar to those discussed herein for the TCH stream). The intermediate bottoms stream may further comprise heavies (amounts similar to those discussed herein for the (second) intermediate adiponitrile stream.

In some case, the intermediate bottoms stream may be further separated, e.g., to yield the bottoms heavies stream and the TCH stream.

### Heavies Stream

As a result of the disclosed operation parameters, in some embodiments, the heavies stream, which may, in some cases be a bottoms stream from a second column of a two column system, may comprise high amounts of TCH as well as heavies.

The heavies stream comprises high-boiling components (heavies). In one embodiment, the heavies stream comprises high-boiling components in an amount ranging from 0.1 wt % to 50 wt %, e.g., from 0.1 wt. % to 20 wt. %, from 0.1 wt. % to 10 wt. %, from 0.5 wt. % to 10 wt. %, from 0.5 wt. % to 5 wt. %, from 1 wt. % to 3 wt. %, from 5 wt. % to 50 wt. %, e.g., from 5 wt. % to 45 wt. %, from 5 wt. % to 40 wt. %, from 5 wt. % to 35 wt. %, from 5 wt. % to 30 wt. %, from 8 wt. % to 50 wt. %, from 8 wt. % to 45 wt. %, from 8 wt. % to 40 wt. %, from 8 wt. % to 35 wt. %, from 8 wt. % to 30 wt. %, from 10 wt. % to 50 wt. %, from 10 wt. % to 45 wt. %, from 10 wt. % to 40 wt. %, from 10 wt. % to 35 wt. %, from 10 wt. % to 30 wt. %, from 12 wt. % to 50 wt. %, from 12 wt. % to 45 wt. %, from 12 wt. % to 40 wt. %, from 12 wt. % to 35 wt. %, from 12 wt. % to 30 wt. %, from 15 wt. % to 50 wt. %, from 15 wt. % to 45 wt. %, from 15 wt. % to 40 wt. %, from 15 wt. % to 35 wt. %, or from 15 wt. % to 30 wt. %. In terms of upper limits, the heavies stream may comprise less than 50 wt. % high-boiling components, e.g., less than 45 wt. %, less than 40 wt. %, less than 35 wt. %, less than 30 wt. %, less than 20 wt. %, less than 10 wt. %, less than 5 wt. %, or less than 3 wt. %. In terms of lower limits, the second bottom heavies stream may comprise greater than 0.1 wt. % high-boiling components, e.g., greater than 0.5 wt %, greater than 1 wt. %, greater than 5 wt. %, greater than 8 wt. %, greater than 10 wt. %, greater than 12 wt. %, or greater than 15 wt. %.

In some cases, the heavies stream may comprise TCH in amounts ranging from 90 wt. % to 100 wt. %, e.g., from 90 wt. % to 99.9 wt. %, from 90 wt. % to 99 wt. %, from 90 wt. % to 98 wt. %, from 92.5 wt. % to 100 wt. %, from 92.5 wt. % to 99.9 wt. %, from 92.5 wt. % to 99 wt. %, from 92.5 to 98 wt. %, from 95 wt. % to 100 wt. %, from 95 wt. % to 99.9 wt. %, from 95 wt. % to 99 wt. %, from 95 to 98 wt. %, from 97.5 wt. % to 100 wt. %, from 97.5 wt. % to 99.9 wt. %, from 97.5 to 99 wt. %, or from 97.5 to 98 wt. %. In terms of upper limits, the heavies stream may comprise less than 100 wt. % TCH, e.g., less than 99.9 wt. % less than 99 wt. %, or less than 98 wt. %. In terms of lower limits, the heavies stream may comprise greater than 90 wt. %, e.g., greater than 92.5 wt. %, greater than 95 wt. %, or greater than 97.5 wt. %.

In some embodiments, the heavies stream may comprise low amounts of lights and/or adiponitrile. For example, the heavies stream may comprise lights and/or adiponitrile in amounts similar to those discussed above with respect to the intermediate bottoms stream or the TCH stream. The heavies stream may further comprise heavies in amounts similar to those discussed herein for the adiponitrile stream.

### Purification

In some cases, the first TCH stream is further purified to yield the purified TCH stream. In some cases, the purification comprises purification in one or more columns. The column operation may be as has been disclosed with respect to the first column, e.g., at low pressure drops and/or with high efficiency packing material. And the noted benefits will accompany these operations.

In some cases, the purified TCH stream comprises TCH. In one embodiment, the purified TCH stream comprises TCH in an amount ranging from 90 wt. % to 100 wt. %, e.g., from 90 wt. % to 99.9 wt. %, from 90 wt. % to 99 wt. %, from 90 wt. % to 98 wt. %, from 92.5 wt. % to 100 wt. %, from 92.5 wt. % to 99.9 wt. %, from 92.5 wt. % to 99 wt. %, from 92.5 to 98 wt. %, from 95 wt. % to 100 wt. %, from 95 wt. % to 99.9 wt. %, from 95 wt. % to 99 wt. %, from 95 to 98 wt. %, from 97.5 wt. % to 100 wt. %, from 97.5 wt. % to 99.9 wt. %, from 97.5 to 99 wt. %, or from 97.5 to 98 wt. %. In terms of upper limits, the purified TCH stream may comprise less than 100 wt. % TCH, e.g., less than 99.9 wt. % less than 99 wt. %, or less than 98 wt. %. In terms of lower limits, the purified TCH stream may comprise greater than 90 wt. %, e.g., greater than 92.5 wt. %, greater than 95 wt. %, or greater than 97.5 wt. %. Conventional processes have been unable to achieve such high TCH purity levels.

In one embodiment, the purified TCH stream comprises impurities, e.g., adiponitrile, amines, and other impurities, in the amount discussed herein relating to the first TCH stream.

In some cases, the first overhead stream (from the first column) is purified, optionally via one or more distillation columns, to form a purified adiponitrile stream comprising at greater than 50 wt % adiponitrile. In some cases, the intermediate adiponitrile stream may be purified using existing purification equipment outside of the process, e.g., in a separation train for a different process.

In some embodiments, the purified adiponitrile stream comprises greater than 10 wt % adiponitrile, e.g., greater than 25 wt %, greater than 50 wt %, greater than 75 wt %, greater than 90 wt %, greater than 92 wt %, greater than 95 wt %, or greater than 97 wt %. In terms of ranges, the purified adiponitrile stream may comprise from 50 wt % to 100 wt % adiponitrile, e.g., from 50 wt % to 99.5 wt %, from 65 wt % to 99 wt %, from 75 wt % to 99 wt %, from 90 wt % to 97 wt %, or from 90 wt % to 95 wt %.

In some cases, both the purified adiponitrile stream and the TCH stream exist (as described herein). In some embodiments, the purified adiponitrile stream comprises greater than 95 wt % adiponitrile and the TCH stream comprises greater than 95 wt % TCH.

In some cases, the purification of the first overhead stream may be conducted in an outside system, e.g., a refinement process, for example in an adiponitrile production process.

### Decomposition

As noted above, the inventors now have found that, in conventional TCH purification processes, certain high-boiling components are prone to decomposition into impurities having both higher boiling points and/or lower boiling points. The inventors have also found that even TCH can decompose at high pressures and/or temperatures in conventional processes. In particular, the inventors have now found that prolonged exposure to high pressures and/or temperatures, such as in columns, contributes to the decomposition of high-boiling components. By utilizing the specific process parameters disclosed herein, this decomposition can be effectively mitigated.

Conventional processes typically require the exposing process streams to high temperatures due to the presence of high-boiling components. TCH, for example, of about 407° C. at atmospheric pressure. As can be appreciated by those skilled in the art, purification of TCH therefore conventionally requires exposing process streams to high temperatures, e.g., at least 350° C., at least 375° C., at least 400° C., or at least 410° C. At these high temperatures, however, the present inventors have found that high-boiling components, such as TCH and adiponitrile, rapidly decompose. As a result, conventional processes experience high inefficiencies. By utilizing the specific process parameters disclosed herein, however, this decomposition can be effectively mitigated or eliminated.

In one aspect, the purification process may inhibit decomposition by reducing the residence time during which process streams are exposed to high temperatures, e.g., in a separation operation. Generally, process streams may be exposed to high temperatures and/or pressures in a column. In order to reduce prolonged exposure, the process may reduce the residence time of a stream in a given column. For example, the process may control the residence time of the adiponitrile process stream (or another purification stream) in a column. In one embodiment, the process limits the residence time of the adiponitrile process stream (or another purification stream) in a column to less than 8 hours, e.g., less than 7 hours, less than 6 hours, less than 5 hours, or less than 4 hours.

In some aspects, the purification processes may inhibit decomposition by reducing the exposure of process streams to high pressures and/or pressure drops. For example, the process may control the pressure to which the adiponitrile process stream (or another purification stream) is exposed, e.g., in the separation step. In one embodiment, the purification process limits the pressure at which separation step(s) are conducted. For example, operation pressure may be limited to less than 50 torr, e.g., less than 45 torr, less than 40 torr, less than 35 torr, less than 30 torr, or less than 25 torr. In order to reduce prolonged exposure to high pressures, the process may reduce the residence time of a stream in a given column. For example, the process may control the residence time of the adiponitrile process stream in a high-pressure column (e.g., a column with a pressure greater than 50 torr).

In some aspects, the purification processes may inhibit decomposition by operating one or more (e.g., all) distillation columns in the second separating step at reduced pressure. At lower pressures, the boiling points of the high-boiling components are reduced, allowing for effective separation of the process streams with exposure to high temperatures. Said another way, at least one of the distillation columns of the second separating is a low pressure distillation column. In one embodiment, the low pressure distillation column(s) is operated with a column top pressure less than 100 mm Hg, e.g., less than 80 mm Hg, less than 60 mm Hg, less than 40 mm Hg, less than 20 mm Hg, less than 15 mm Hg, less than 10 mmHg, less than 5 mm Hg, or less than 3 mm Hg. In one embodiment, the low pressure distillation column(s) is operated with a column bottom pressure less than 100 mm Hg, e.g., less than 80 mm Hg, less than 60 mm Hg, less than 40 mm Hg, less than 20 mm Hg, less than 15 mm Hg, less than 10 mmHg, less than 5 mm Hg, or less than 3 mm Hg. In one embodiment, the low pressure distillation column(s) is operated under vacuum.

In one aspect, the separation and/or purification steps may inhibit decomposition by reducing the exposure of process streams to high temperatures. For example, the process may control the temperature to which the adiponitrile process stream (or another purification stream) is exposed, e.g., in a separation step. In one embodiment, the purification process limits the temperature at which separation step(s) are conducted. For example, operation temperature may be limited to less than 350° C., e.g., less than 325° C., less than 300° C., less than 275° C., or less than 250° C., In terms of ranges operation temperature may range from 225° C. to 350° C., e.g., from 250° C. to 325° C. or from 275° C. to 300° C., or from 250° C. to 275° C.

In some aspects, the process may control both the temperature to which a stream is exposed and the time for which it is exposed to that temperature. For example, the process may control the residence time of the adiponitrile process stream (or another purification stream) in a column as well as the temperature of that distillation column. In one embodiment, the residence time of a stream in temperatures above 230° C. is less than 8 hours. The aforementioned ranges and limits for temperature and residence time may be combined with one another.

In some aspects, the process may control both the temperature to which a stream is exposed and the pressure to which it is exposed. In one embodiment, the process may be controlled such that the stream is not exposed to temperatures above 300° C. or pressures above 35 torr.

In other aspects, the process may inhibit decomposition by utilizing columns with certain physical features. In particular, the distillation columns employed in the purification process may have certain shapes. In some embodiments, the distillation columns have relatively small sumps to minimize exposure to high temperatures. In these embodiments, the sumps of each column may taper to a smaller diameter, which allows or reduced exposure to higher temperatures.

To effectively operate at such high temperatures, the reboiler may require special systems. In some embodiments, the reboiler utilizes a hot oil system sufficient to support high temperatures. Those of skill in the art will appreciate how to utilize a hot oil system in accordance with the processes described herein.

These modifications to conventional purification processes reduce the decomposition of high-boiling components. In some embodiments, these modifications reduce the amount high-boiling components in the first overhead stream that decompose during the second separating step. In one embodiment, the amount of high-boiling components in the adiponitrile process stream (or another purification stream) that decompose is less than 50 wt. % of the high-boiling components in the stream, e.g., less than 45 wt. %, less than 40 wt. %, or less than 30 wt. %. In terms of lower limits, the amount of high-boiling components that decompose may be greater than 0 wt. % of the high-boiling components in the stream, e.g., greater than 5 wt. %, greater than 10 wt. %, or greater than 15 wt. %. In terms of ranges, the amount of high-boiling components that decompose may be from 0 wt. %. to 50 wt. %, e.g., from 0 wt. % to 45 wt. %, from 0 wt. % to 40 wt. %, from 0 wt. % to 30 wt. %, from 5 wt. % to 50 wt. %, from 5 wt. % to 45 wt. %, from 5 wt. % to 40 wt. %, from 5 wt. % to 30 wt. %, from 10 wt. % to 50 wt. %, from 10 wt. % to 45 wt. %, from 10 wt. % to 40 wt. %, from 10 wt. % to 30 wt. %, from 15 wt. % to 50 wt. %, from 15 wt. % to 45 wt. %, from 15 wt. % to 40 wt. %, or from 15 wt. % to 30 wt. %.

In some embodiments, the various process streams individually comprise less than 1 wt. % decomposition products of high-boiling components, e.g., less than 0.8 wt. %, less than 0.5 wt. %, less than 0.3 wt. %, less than 0.1 wt. %, less than 0.05 wt. %, or less than 0.01 wt. %.

As noted above, the high-boiling components may decompose into other high-boiling impurities and/or into low-boiling impurities. In some cases, the high-boiling components may decompose into other high-boiling impurities that were not otherwise present in the system. Said another way, the decomposition may cause the total number of high-boiling impurity compounds in the system to increase. By inhibiting decomposition, as described herein, the increase in the total number of high-boiling impurity compounds present in the system, caused by decomposition, may be reduced.

In some cases, the column(s) may operate with a short residence time. The residence time of feed streams in the individual separation and/or purification operations of the process is minimized, e.g., less than 8 hours, e.g., less than 7 hours, less than 6 hours, less than 5 hours, or less than 4 hours. The lower residence times (optionally in combination with the lower pressure drop) unexpectedly contributes to the separation/purification efficiencies.

### Recycle Step

In some embodiments, the process comprises a recycle step of recycling at least a portion of a (bottoms) stream formed during the separation steps to a point upstream (target). For example, the recycling step may comprise recycling at least a portion of the heavies stream of one of the columns or flashers to a point upstream in the process. In some embodiments, the recycling step comprises recycling at least a portion of the heavies stream of the separation step to the flasher overhead stream of the flashing step. In some embodiments, the recycling step comprises recycling at least a portion of a bottoms stream of the purification step to the flasher overhead stream of the flashing step and/or the bottoms stream of the separation step.

In one embodiment, the recycled stream comprises heavies, and the concentration of these heavies surprisingly affects the purity of the resultant TCH stream and may help to control the concentration of high-boiling components in the overhead streams to be from 0 wt. % to 10 wt. %. In some cases, the concentration of high-boiling components in the recycle streams leads to lesser amounts of high-boiling components in the various overhead streams, which in turn leads to higher purity of adiponitrile and/or TCH.

In some cases, the recycled stream comprises heavies in an amount ranging from 0 wt. % to 40 wt. %, e.g., from 0 wt. % to 37.5 wt. %, from 0 wt. % to 35 wt. %, from 0 wt. % to 32.5 wt. %, from 0 wt. % to 30 wt. %, from 5 wt. % to 40 wt. %, from 5 wt. % to 37.5 wt. %, from 5 wt. % to 35 wt. %, from 5 wt. % to 32.5 wt. %, from 5 wt. % to 30 wt. %, from 10 wt. % to 40 wt. %, from 10 wt. % to 37.5 wt. %, from 10 wt. % to 35 wt. %, from 10 wt. % to 32.5 wt. %, from 10 wt. % to 30 wt. %, from 15 wt. % to 40 wt. %, from 15 wt. % to 37.5 wt. %, from 15 wt. % to 35 wt. %, from 15 wt. % to 32.5 wt. %, from 15 wt. % to 30 wt. %, from 20 wt. % to 40 wt. %, from 20 wt. % to 37.5 wt. %, from 20 wt. % to 35 wt. %, from 20 wt. % to 32.5 wt. %, or from 20 wt. % to 30 wt. %. In terms of upper limits, the recycled stream may comprise less than 40 wt. % high-boiling components, e.g., less than 37.5 wt. %, less than 35 wt. %, less than 32.5 wt. %, or less than 30 wt. %. In terms of lower limits, the recycled stream may comprise greater than 0 wt. % high-boiling components, e.g., greater than 5 wt. %, greater than 10 wt. %, greater than 15 wt. %, or greater than 20 wt. %.

In some aspects, the recycle step controls the concentration of heavies in the target. For example, the recycle step may control the concentration of the heavies in the flasher overhead stream by recycling a stream containing heavies to the flasher stream.

In one embodiment, due to the recycling, the recycle step controls the concentration of heavies in the target to be from 0 wt. % to 10 wt. %, e.g., from 0 wt. % to 9 wt. %, from 0 wt. % to 8 wt. %, from 0 wt. % to 7 wt. %, from 1 wt. % to 10 wt. %, from 1 wt. % to 9 wt. %, from 1 wt. % to 8 wt. %, from 1 wt. % to 7 wt. %, from 2 wt. % to 10 wt. %, from 2 wt. % to 9 wt. %, from 2 wt. % to 8 wt. %, from 2 wt. % to 7 wt. %, from 3 wt. % to 10 wt. %, from 3 wt. % to 9 wt. %, from 3 wt. % to 8 wt. %, or from 3 wt. % to 7 wt. %. In terms of upper limits, the recycle step may control the concentration of heavies in the target to be less than 10 wt. %, e.g., less than 9 wt. %, less than 8 wt. %, or less than 7 wt. %. In terms of lower limits, the recycle step may control the concentration of heavies in the target to be greater than 0 wt. %, e.g., greater than 1 wt. %, greater than 2 wt. %, or greater than 3 wt. %.

### Treating Step

As noted above, the first TCH stream produced in the separating step may comprise impurities. These impurities may be removed by further purification methods. In some embodiments, the purification process further comprises a treating step of treating the first TCH stream to form the purified TCH stream.

In some embodiments, the treating step may comprise nitrogen stripping. In some embodiments, the treating step may comprise treating with one or more types of molecular sieve. In some embodiments, the treating step may comprise a combination of treating with nitrogen stripping and treating with molecular sieves.

The purified TCH stream comprises a lower concentration of impurities than that of the first TCH stream. In one embodiment, the purified TCH stream comprises less than 0.1 wt. %, impurities, e.g., less than 0.09 wt. %, less than 0.05 wt. %, or less than 0.01 wt. %. For example, the purified TCH stream may comprise water as an impurity. In one embodiment, the purified TCH stream comprises less than 20 ppm water, e.g., less than 15 ppm, less than 10 ppm, or less than 1 ppm. The purified TCH stream may comprise metals as impurities. In one embodiment, the purified TCH stream comprises less than 5 ppm metals, e.g., less than 4 ppm, less than 3 ppm, or less than 2 ppm.

Exemplary separation and/or purification schemes are disclosed in U.S. Provisional Patent No. 62/852,604, filed on May 24, 2019, the contents of which are incorporated by reference herein.

### Configurations

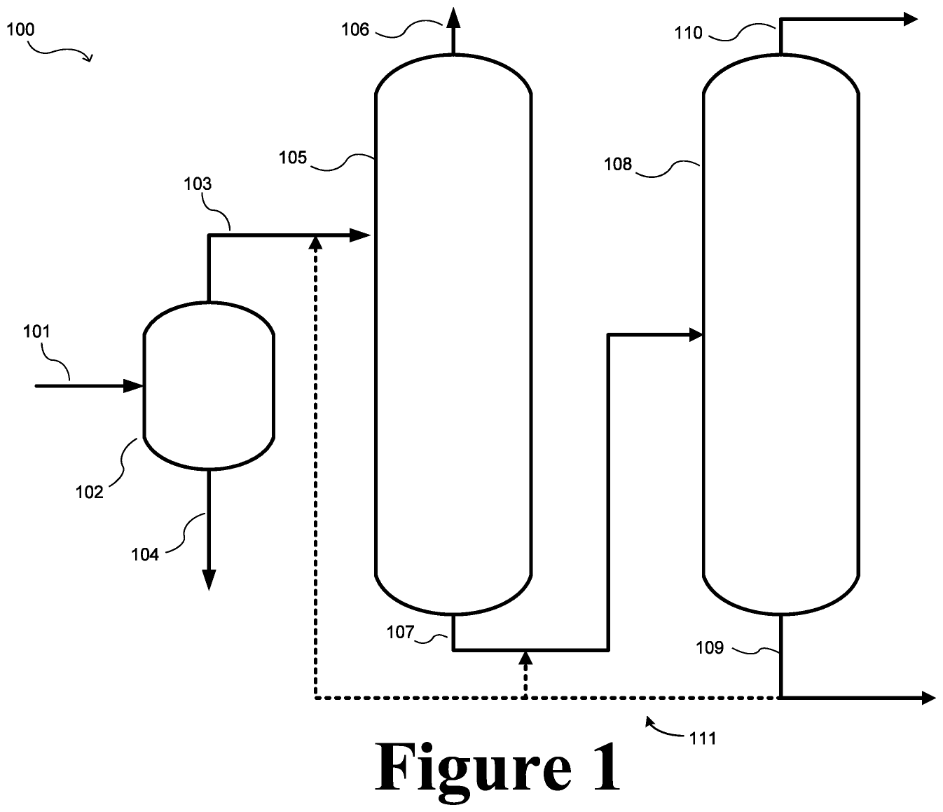

FIGS. 1-5 show schematic overviews of several configurations of the TCH purification processes disclosed herein.

FIG. 1 shows one embodiment of the adiponitrile separation process 100. In this embodiment, an adiponitrile process stream 101 is separated in a flash evaporator 102 to form a first overhead stream 103 and a first bottoms stream 104. The first overhead stream 103 is then separated in a first distillation column 105 to form a lights stream as a second overhead stream 106 and a second bottoms stream 107. The second bottoms stream is then separated in a second distillation column 108 to form a heavies stream as a third bottoms stream 109 and a TCH stream as a third overhead stream 110. This embodiment also features an optional recycle step 111, whereby a portion of the third bottoms stream 109 is recycled to the first overhead stream 103 and/or the second bottoms stream 107.

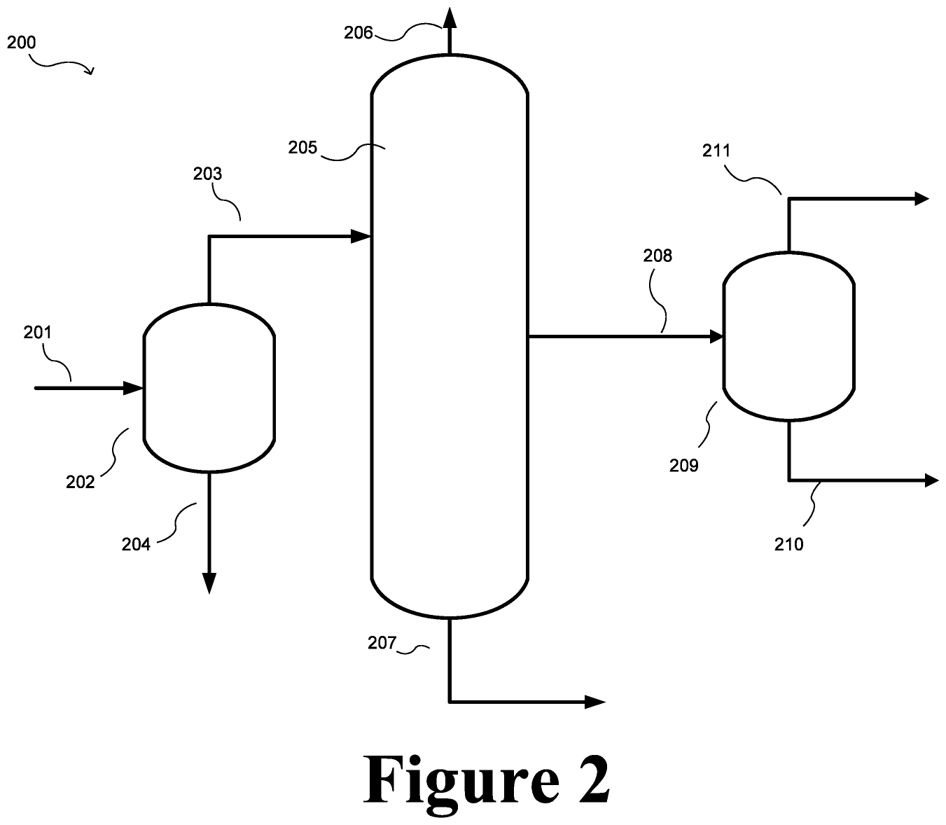

FIG. 2 shows another embodiment of the adiponitrile separation process 200. In this embodiment, an adiponitrile process stream 201 is separated in a flash evaporator 202 to form a first overhead stream 203 and a first bottoms stream 204. The first overhead stream 203 is then separated in a first distillation column 205 to form a lights stream as a second overhead stream 206, a second bottoms stream 207, and a side draw 208. The side draw 208 is then separated in separated in a flasher 209 to form a TCH stream as a third bottoms stream 210 and a third overhead stream 211.

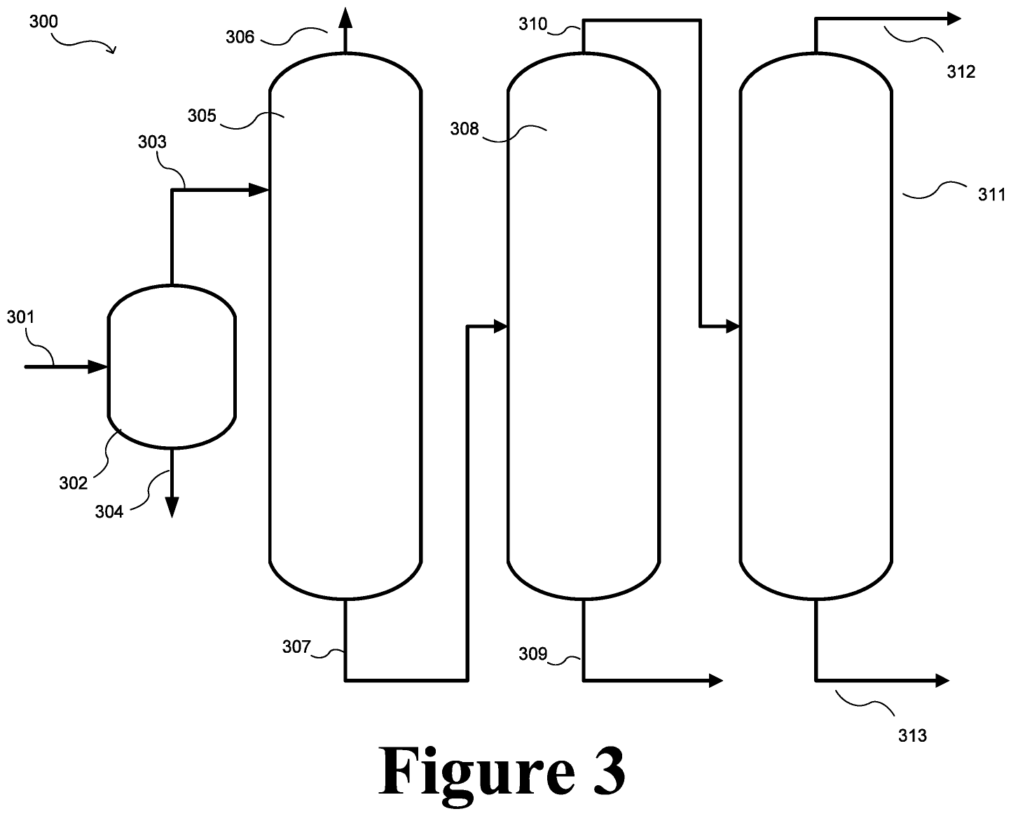

FIG. 3 shows another embodiment of the adiponitrile separation process 300. In this embodiment, an adiponitrile process stream 301 is separated in a flash evaporator 302 to form a first overhead stream 303 and a first bottoms stream 304. The first overhead stream 303 is then separated in a first distillation column 305 to form a lights stream as a second overhead stream 306 and a second bottoms stream 307. The second bottoms stream 307 is then separated in a second distillation column 308 to form a heavies stream as a third bottoms stream 309 and a third overhead, or distillate, stream 310. The third overhead stream 310 is then separated in a third distillation column 311 to form a fourth overhead stream 312 and a TCH stream as a fourth bottoms stream 313.

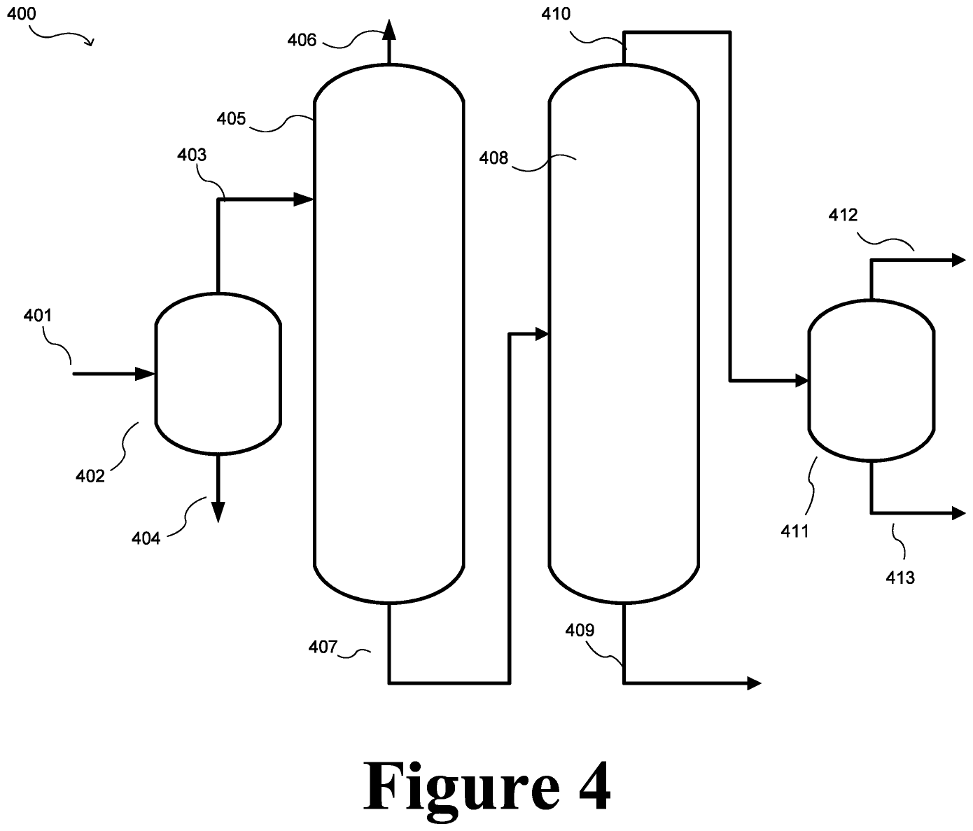

FIG. 4 shows another embodiment of the adiponitrile separation process 400. In this embodiment, an adiponitrile process stream 401 is separated in a flash evaporator 402 to form a first overhead stream 403 and a first bottoms stream 404. The first overhead stream 403 is then separated in a first distillation column 405 to form a lights stream as a second overhead stream 406 and a second bottoms stream 407. The second bottoms stream 407 is then separated in a second distillation column 408 to form a heavies stream as a third bottoms stream 409 and a third overhead, or distillate, stream 410. The third overhead stream 410 is then separated in a flasher 411 to form a fourth overhead stream 412 and a TCH stream as a fourth bottoms stream 413.

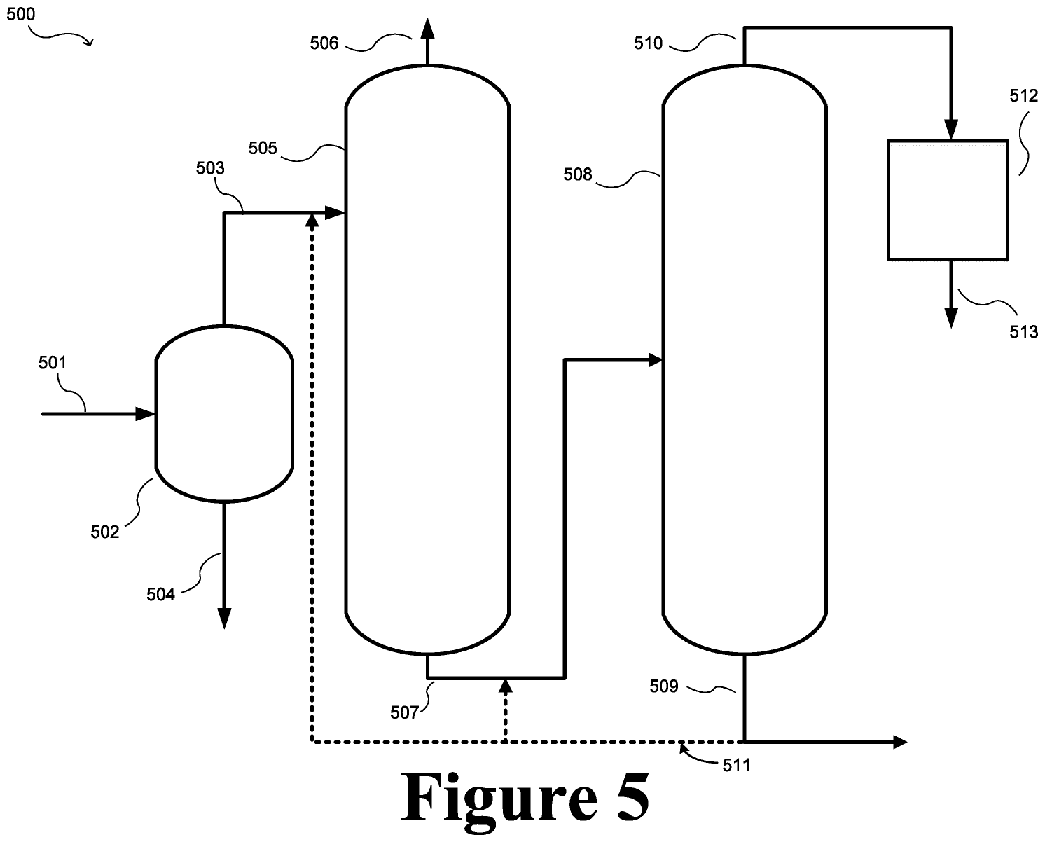

FIG. 5 shows another embodiment of the adiponitrile separation process 500. In this embodiment, an adiponitrile process stream 501 is separated in a flash evaporator 502 to form a first overhead stream 503 and a first bottoms stream 504. The first overhead stream 503 is then separated in a first distillation column 505 to form a lights stream as a second overhead stream 506 and a second bottoms stream 507. The second bottoms stream 507 is then separated in a second distillation column 508 to form a heavies stream as a third bottoms stream 509 and a TCH stream as a third overhead stream 510. This embodiment also features an optional recycle step 511, whereby a portion of the third bottoms stream 509 is recycled to the first overhead stream 503 and/or the second bottoms stream 507. This embodiment also features a treating step 512, whereby the TCH stream 510 is subjected to further treatment to yield a purified TCH stream 513.

The present disclosure will be further understood by reference to the following non-limiting example.

### EXAMPLES

For Examples 1 and 2, a crude adiponitrile process stream was collected from an adiponitrile production and purification process. The crude adiponitrile process streams of Examples 1 and 2 were fed to a separation process as described herein, e.g., similar to the separation described in FIG. 1.

The adiponitrile process streams were separated in a wiped film evaporator multiple times times, e.g., two or four times. The multiple passes through the wiped film evaporator produced an overhead (adiponitrile process stream) and first bottoms heavies stream, which comprised high-boiling components and solid impurities. The first bottoms stream was discarded. The compositions of the adiponitrile process stream and the first bottoms stream are provided in

TABLE 1
TCH content, in some cases, included TCH and isomers thereof.
Table 1: First Separating Step Flash

| | Adiponitrile Process | Bottoms Stream | |
|---|---|---|---|
| Component | Stream | Ex. 1 | Ex. 2 |
| Adiponitrile | 5.0 | 1.0 | 0.7 |
| TCH | 80.0 | 95.0 | 95.9 |
| Lights | 5.0 | 1.5 | 1.8 |
| Heavies | 10.0 | 2.5 | 2.4 |

The adiponitrile process streams of Examples 1 and/or 2 were distilled in a first column comprising high efficiency packing. The first distillation column was operated at a column bottom temperature of about 255° C., and at 1 mmHg and the residence time of the first overhead lights stream in the first distillation column was less than 4 hours. Pressure drop across the column was less than 11 mmHg, e.g., 10 mmHg or 7 mmHg. The first column produced an adiponitrile stream, which was beneficially enriched in adiponitrile. Samples of this stream were collected at various times and analyzed. Compositions of these samples are shown in Table 2a. At various times, pressure drop across the column ranged from 1 mmHg to 11 mmHg, e.g., from 5 mmHg to 7 mmHg. The column was packed with high efficiency packing. And the pressure drop per theoretical stages of the column, at various times, ranged from 0.01 mmHg to 1.5 mmHg, e.g., from 0.3 mmHg to 0.6 mmHg. In some cases, the number of cycles in the wiped film evaporator was found to affect the composition of the resulting overhead.

TABLE 2a
Separating Step (First Column)

| | Adiponitrile Stream | | |
|---|---|---|---|
| Component | Sam. 1 | Sam. 2 | Sam. 3 |
| Adiponitrile | 7.1 | 27.09 | 8.93 |
| TCH | 80.3 | 45.77 | 70.49 |
| Lights | 10.6 | 24.59 | 18.97 |
| Heavies | 2.0 | 2.54 | 2.27 |

The distillation column also produced a bottoms stream, which contained a high concentration of TCH and some heavies. Samples of this stream were collected at various times and analyzed. Compositions of these samples are shown in Table 2b.

TABLE 2b
Second Separating Step (First Column)

| | Second Bottoms Stream | | | | | | | | | | |
|---|---|---|---|---|---|---|---|---|---|---|---|
| | Sam. | Sam. | Sam. | Sam. | Sam. | Sam. | Sam. | Sam. | Sam. | Sam. | Sam. |
| Comp. | 4 | 5 | 6 | 7 | 8 | 9 | 10 | 11 | 12 | 13 | 14 |
| Adipo | 0.0 | 0.009 | 0 | 0 | 0.003 | 0.006 | 0.004 | 0 | 0 | 0 | 0 |
| TCH | 97.4 | 97.57 | 96.21 | 97.24 | 97.5 | 97.4 | 97.4 | 97.3 | 97.8 | 97.7 | 98.1 |
| Lights | 0.0 | 0.21 | 0.04 | 0.14 | 0.19 | 0.09 | 0.1 | 0.11 | 0.05 | 0.00 | 0.03 |
| Heavies | 2.6 | 2.2 | 3.75 | 2.62 | 2.30 | 2.53 | 2.53 | 2.48 | 2.18 | 2.34 | 1.9 |

The second bottoms streams were then distilled in a second distillation column. The second distillation column was operated at a column bottom temperature of about 263° C., an operating pressure of about 1 mmHg, and the residence time of the second bottoms stream in the second distillation column was less than 4 hours. The second distillation column produced a third bottoms stream (heavies stream). The heavies stream can be recycled and/or discarded. The second distillation column also produced a third overhead stream (TCH stream). The column was packed with high efficiency packing. At various times, pressure drop across the second column and the pressure drop per theoretical stages of the second column, at various times, were similar to those of the first column (see above). Samples of these streams were collected at various times and analyzed. Compositions of these samples are shown in Tables 3a-3d.

TABLE 3a
Separating Step (Second Column)

| | TCH Stream | | | | | | | |
|---|---|---|---|---|---|---|---|---|
| Component | Sam. 15 | Sam. 16 | Sam. 17 | Sam. 18 | Sam. 19 | Sam. 20 | Sam. 21 | Sam. 22 |
| Adiponitrile | 0.108 | 0.071 | 0.129 | 0.045 | 0.051 | 0.12 | 0.05 | 0.02 |
| TCH | 98.88 | 98.95 | 98.77 | 97.0 | 97.72 | 98.18 | 99.21 | 99.0 |
| Lights | 0.34 | 0.27 | 0.29 | 0.30 | 0.29 | 0.34 | 0.23 | 0.08 |
| Heavies | 0.67 | 0.67 | 0.81 | 2.61 | 1.89 | 1.34 | 0.51 | 0.89 |

TABLE 3b
Separating Step (Second Column)

| | TCH Stream | | | | | | | |
|---|---|---|---|---|---|---|---|---|
| Component | Sam. 22 | Sam. 23 | Sam. 24 | Sam. 25 | Sam. 26 | Sam. 27 | Sam. 28 | Sam. 29 |
| Adiponitrile | 0.046 | 0.026 | 0.021 | 0.016 | 0.03 | 0.02 | 0.038 | 0.023 |
| TCH | 99.12 | 99.06 | 99.03 | 99.65 | 99.03 | 99.28 | 99.34 | 99.48 |
| Lights | 0.46 | 0.14 | 0.08 | 0.11 | 0.22 | 0.16 | 0.12 | 0.19 |
| Heavies | 0.38 | 0.77 | 0.87 | 0.22 | 0.72 | 0.55 | 0.53 | 0.30 |

TABLE 3c
Separating Step (Second Column)

| | Heavies Stream | | | | | | | |
|---|---|---|---|---|---|---|---|---|
| Component | Sam. 30 | Sam. 31 | Sam. 32 | Sam. 33 | Sam. 34 | Sam. 35 | Sam. 36 | Sam. 37 |
| Adiponitrile | 0 | 0 | 0 | 0 | 0 | 0 | 0 | 0 |
| TCH | 90.92 | 95.36 | 93.08 | 95.0 | 94.29 | 94.52 | 97.32 | 97.23 |
| Lights | 0.07 | 0.06 | 0.11 | 0.11 | 0.07 | 0.02 | 0.11 | 0.07 |
| Heavies | 9.01 | 4.55 | 6.81 | 4.9 | 5.63 | 5.46 | 2.48 | 2.7 |

TABLE 3d
Separating Step (Second Column)

| | Heavies Stream | | | | | | |
|---|---|---|---|---|---|---|---|
| Com- | Sam. | Sam. | Sam. | Sam. | Sam. | Sam. | Sam. |
| ponent | 38 | 39 | 40 | 41 | 42 | 43 | 44 |
| Adi- | 0 | 0 | 0 | 0 | 0 | 0 | 0 |
| ponitrile | | | | | | | |
| TCH | 94.99 | 92.43 | 91.76 | 89.65 | 90.27 | 88.56 | 95.06 |
| Lights | 0.09 | 0 | 0 | 0 | 0 | 0 | 0 |
| Heavies | 4.91 | 7.56 | 8.24 | 10.35 | 9.73 | 11.44 | 4.94 |

As the above tables show, the purification process carried out in Examples 1 and 2, with low column pressure drop, e.g., less than 25 mm Hg or less than 11 mmHg or from 1 mmHg to 11 mmHg and/or at a pressure drop per theoretical stages of the column ranging from 0.01 mmHg to 1.5 mmHg, produced a highly pure TCH stream. In particular, the purification process resulted in a TCH stream comprising greater than 97 wt. % TCH, e.g., in most cases greater than 99 wt. %, and comprising little or no measurable adiponitrile or lights. As shown, the concentration of the heavies in the second bottoms stream and/or the heavies stream was maintained within the ranges and limits disclosed herein.

In addition, the separation process produced an adiponitrile stream in (first column overhead) which the adiponitrile concentration was improved over the initial adiponitrile concentration in the feed.

As shown, it was unexpectedly found that as the feed to the column(s) has a higher adiponitrile concentration, the concentration improvement in the column overhead is surprisingly improved. In simulations using similar equipment, when adiponitrile concentration in the column feed was above 10 wt %, then the adipo concentration in the overhead was advantageously higher, e.g., over 50%.

### EMBODIMENTS

The following embodiment, among others, are disclosed.

Embodiment 1: A process for producing a TCH stream, the process comprising: separating, in a first column, an adiponitrile process stream comprising TCH and optionally adiponitrile, to form an adiponitrile stream comprising greater than 5 wt. % adiponitrile and a first TCH stream comprising TCH, and optionally a heavies stream comprising high-boiling components and solid impurities; and optionally purifying the first TCH stream, via one or more columns, to form a purified TCH stream comprising greater than 50 wt. % TCH; wherein the first column is operated at a pressure drop less than 25 mmHg.

Embodiment 2: an embodiment of embodiment 1, wherein the first column is a packed column and the packing comprises high efficiency packing, wherein the high efficiency packing provides for a pressure drop of less than 0.5 mmHg/theoretical stage.

Embodiment 3: an embodiment of embodiment 1 or 2, wherein the purified TCH stream comprises less than 1 wt. % impurities.

Embodiment 4: an embodiment of any of embodiments 1-3, wherein the purified TCH stream comprises less than 1 wt. % decomposition products of high-boiling components.

Embodiment 5: an embodiment of any of embodiments 1-4, wherein the purified TCH stream comprises less than 1 wt. % amines.

Embodiment 6: an embodiment of any of embodiments 1-5, further comprising: flashing a crude adiponitrile stream to form the adiponitrile process stream and a bottoms stream comprising high-boiling components and solid impurities.

Embodiment 7: an embodiment of any of embodiments 1-6, wherein the crude adiponitrile stream comprises less than 25 wt. % TCH.

Embodiment 8: an embodiment of any of embodiments 1-7, wherein the purifying comprises: separating, in a second column, the first TCH stream to form the purified TCH stream and a heavies stream comprising high-boiling components.

Embodiment 9: an embodiment of any of embodiments 1-8, wherein residence time is less than 8 hours.

Embodiment 10: an embodiment of any of embodiments 1-9, wherein the first and second columns are operated at a pressure drop less than 25 mmHg.

Embodiment 11: an embodiment of any of embodiments 1-10, wherein the second column is a packed column and the packing comprises high efficiency packing.

Embodiment 12: an embodiment of any of embodiments 1-11, wherein the TCH stream comprises: TCH, from 0 wt. % to 0.05 wt. % adiponitrile, from 0 wt. % to 0.1 wt. % di(2-cyanoethyl) amine, from 0 wt. % to 0.05 wt. % cyanovaleramide, and from 0 wt. % to 0.05 wt. % tri(2-cyanoethyl) amine.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit.

1. A process for producing a TCH stream, the process comprising:
separating, in a first column, an adiponitrile process stream comprising TCH and optionally adiponitrile, to form an adiponitrile stream comprising greater than 5 wt. % adiponitrile and a first TCH stream comprising TCH, and optionally a heavies stream comprising high-boiling components and solid impurities; and
optionally purifying the first TCH stream, via one or more columns, to form a purified TCH stream comprising greater than 50 wt. % TCH;
wherein the first column is operated at a pressure drop less than 25 mmHg.

2. The process of claim 1, wherein the first column is a packed column and the packing comprises high efficiency packing, wherein the high efficiency packing provides for a pressure drop of less than 0.5 mmHg/theoretical stage.

3. The process of claim 1, wherein the purified TCH stream comprises less than 1 wt. % impurities.

4. The process of claim 1, wherein the purified TCH stream comprises less than 1 wt. % decomposition products of high-boiling components.

5. The process of claim 1, wherein the purified TCH stream comprises less than 1 wt. % amines.

6. The process of claim 1, further comprising:
flashing a crude adiponitrile stream to form the adiponitrile process stream and a bottoms stream comprising high-boiling components and solid impurities.

7. The process of claim 1, wherein the crude adiponitrile stream comprises less than 25 wt. % TCH.

8. The process of claim 1, wherein the purifying comprises:
separating, in a second column, the first TCH stream to form the purified TCH stream and a heavies stream comprising high-boiling components.

9. The process of claim 1, wherein residence time is less than 8 hours.

10. The process of claim 1, wherein the first and second columns are operated at a pressure drop less than 25 mmHg.

11. The process of claim 1, wherein the second column is a packed column and the packing comprises high efficiency packing.

12. The process of claim 1, wherein the TCH stream comprises:
TCH,
from 0 wt. % to 0.05 wt. % adiponitrile,
from 0 wt. % to 0.1 wt. % di(2-cyanoethyl) amine,
from 0 wt. % to 0.05 wt. % cyanovaleramide, and
from 0 wt. % to 0.05 wt. % tri(2-cyanoethyl) amine.
